(19) United States
(12) Patent Application Publication
(10) Pub. No.: US 2026/0000427 A1
(43) Pub. Date: Jan. 1, 2026

(54) SYSTEMS FOR MAKING A FAT-INCLUSIVE SKIN GRAFT TREATMENT COMPOSITION, FAT-INCLUSIVE SKIN GRAFT TREATMENT COMPOSITION, AND METHODS OF MAKING SAME

(71) Applicant: COLOPLAST A/S, Humlebaek (DK)

(72) Inventor: Gudmundur Fertram SIGURJONSSON, Isafjordur (IS)

(21) Appl. No.: 19/111,941

(22) PCT Filed: Sep. 18, 2023
(86) PCT No.: PCT/IB2023/059249

§ 371 Date: Mar. 14, 2025

Related U.S. Application Data
(60) Provisional application No. 63/407,561, filed on Sep. 16, 2022.

(51) Int. Cl. A61B 17/322; A61B 17/00

(52) U.S. Cl. CPC A61B 17/322; A61B 2017/00969; A61B 2017/3225

(57) ABSTRACT

A system for preparing a wound treatment product is provided including an applicator configured to apply a fat material to a skin graft material. A wound treatment kit is provided including a skin graft material and an applicator configured to apply a fat material to the skin graft material. A wound treatment composition is provided including a skin graft material and a fat material, such as to promote cellular regenerative ingrowth into a wound. A method for preparing a wound treatment is provided including providing a fat material; providing a skin graft material; and applying the fat material to the skin graft material. A method for treating a wound is provided including providing a skin graft material; applying a fat material to the skin graft material; and applying the combined fat and skin graft material to the wound.

### TECHNICAL FIELD

The disclosure relates generally to wound treatments, a product for treating wounds for stabilizing, protecting, and/or healing damaged tissue, and methods and systems for making a wound treatment product.

### BACKGROUND

Healthy skin serves several distinct functions, including protecting underlying tissues from abrasion, microbes, water loss, and ultraviolet light damage. The nervous system of healthy, normal skin also provides tactile sensations of touch, pressure, and vibration, thermal sensations of heat and cold, and pain sensations. A body's thermoregulation relies on the skin's ability to sweat and control blood flow to the skin to increase or decrease heat loss. Healthy skin includes three distinct tissue layers: a thin outer layer of cells called the epidermis, a thicker middle layer of connective tissue called the dermis, and an inner, subcutaneous layer. The thin outer layers of the epidermis are composed of flattened, cornified, dead keratinocytes that form a barrier to water loss and microbe entry. The dead keratinocytes are derived from live keratinocytes in the basal layer, which lies above the dermis, and are responsible for skin reepithelization. The epidermis does not contain nerves or blood vessels and obtains water and nutrients through diffusion from the dermis. The dermis, which lies below the epidermis, is composed mostly of collagen fibers and some elastic fibers both produced by fibroblasts and, along with water and large proteoglycan molecules, makes up the extracellular matrix (ECM). This skin layer provides mechanical strength and a substrate for water and nutrient diffusion. It contains blood vessels, nerves, sweat glands, hair follicles, and cells involved in immune function, growth, and repair. The subcutaneous layer is composed of adipocytes that form a thick layer of adipose tissue.

A wound may be considered a disruption of the skin's structural and functional integrity. Thus, a “wound” may include those injuries that cause, for example, cutting, tearing, and/or breaking of the skin such as lacerations, abrasions, incisions, punctures, avulsions, burns, or other such injuries.

After hemostasis, which often follows a wound event, a wound goes through three main stages when healing: inflammation, proliferation and remodeling. Chronic wounds may be considered wounds that have failed to pass through the normal healing process in an orderly and timely manner. Chronic wounds often remain in the inflammation phase.

Often, in the cases of significant wounds, such as wounds extending over large areas or in deep wounds, or large or severe burn wounds, or in the case of chronic wounds, skin substitutes are often used to aid in the healing process of the wound and to more quickly restore at least some of the above-noted functions of healthy skin. Skin substitutes may be considered broadly as a group of elements or materials that enable the temporary or permanent occlusion of a wound. Skin substitutes can generally be divided into biological skin substitutes, synthetic skin substitutes, or a hybrid skin substitute that includes biological and synthetic skin substitutes.

Biological skin substitutes often have a more intact extracellular matrix structure, while the synthetic skin substitutes can be synthesized on demand and can be modulated for specific purposes. Biological skin substitutes and synthetic skin substitutes each have advantages and disadvantages. The biological skin substitutes may allow the construction of a more natural new dermis and allow excellent re-epithelialization characteristics due to the presence of a basement membrane. Synthetic skin substitutes may be chemically synthesized and provide the advantages of increase control over scaffold composition. Synthetic skin substitutes include synthetic biolayers including, for example, a synthesized collagen or protein-based matrix or a collagen or protein-based components combined with silicone components. Hybrid skin substitutes may be partly synthesized or produced by living cells and partly chemically synthesized.

Biological skin substitutes may include, but are not limited to, skin grafts, including autologous skin grafts, syngeneic skin grafts, allogeneic skin grafts, xenogeneic skin grafts such as porcine skin grafts, cadaveric skin allografts, and amniotic tissue grafts.

Whether biological, synthetic, or hybrid skin substitutes are used, the object of using skin substitutes is to provide an effective, timely, and scar-free wound healing with as much return to the functions of the skin before the wound event.

Failure to preserve the tissue conditions of traumatic wounds often leads to tissue desiccation and deterioration. Further, many of the current treatments fail to provide an adequate barrier to subsequent infection and/or fail to confine the wound away from dirt and harmful pathogens. Further, current treatments are ill equipped for adaptation to prolonged field care. In short, there is a need for improved approaches to stabilizing and/or protecting wounds, particularly in the field, to preserve the patient and the wound for further, subsequent care.

In view of the foregoing, the inventors have found there is need for a wound treatment that is rugged and robust, low weight, small, easy to transport and handle, has low dependency on external power or specialized equipment, is modular and interoperable with current approaches to care. There is further a need for a wound treatment that is sustainable, scalable, and safe for human use.

### SUMMARY

A system for preparing a wound treatment is provided comprising an applicator configured to apply a fat material to a skin graft material. An applicator may comprise one or more of a syringe, a plunger, a roller, a screw, a container, a preparation chamber, a loading chamber, an extrusion press, a vacuum source, a mesh screen, an input channel, an output channel, a blade, a frame, a retaining element, and a handle.

A wound treatment kit is provided comprising a skin graft material and an applicator configured to apply a fat material to the skin graft material. The kit may further include one or more of a syringe, a plunger, a roller, a screw, a container, a preparation chamber, a loading chamber, an extrusion press, a vacuum source, a mesh screen, an input channel, an output channel, a blade, a frame, a retaining element, a handle, and sterile packaging.

A wound treatment composition is provided comprising a skin graft material and a fat material, such as to promote cellular regenerative ingrowth into a wound. A fat material may comprise material obtained from a region of a body having fat therein. A skin graft material may comprise a biologic and/or synthetic skin substitute material.

A method for preparing a wound treatment is provided comprising providing a fat material; providing a skin graft material; and applying the fat material to the skin graft material. Applying the fat material to the skin graft material may comprise infiltrating, infusing, impregnating, joining, and/or combining the fat material and the skin graft material.

A method for treating a wound is provided comprising providing a skin graft material; applying a fat material to the skin graft material; and applying the combined fat and skin graft material to the wound.

Further, embodiments of the present disclosure additionally extend to bandages for treatment of a wound. Accordingly, wound treatments, bandages, kits, and methods for stabilizing, protecting, and/or healing a wound are disclosed.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description that follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

These and other features, aspects, and advantages of the present disclosure will become better understood regarding the following description, appended claims, and accompanying drawings.

FIG. 1 is a diagram of a system for preparing a wound treatment in the form of a skin graft material with a fat material according to embodiments of the disclosure.

FIG. 2 is a diagram of an extrusion die for use in a system for preparing a wound treatment in the form of a skin graft material with a fat material according to embodiments of the disclosure.

FIG. 3 is a diagram of a system for preparing a wound treatment in the form of a skin graft material with a fat material according to embodiments of the disclosure.

FIG. 4 is a diagram of a preparation chamber according to embodiments of the disclosure.

FIG. 5 is a diagram of a method for preparing a wound treatment in the form of a skin graft material with a fat material according to embodiments of the disclosure.

FIG. 6A is a diagram of a system for preparing a wound treatment in the form of a skin graft material with a fat material according to embodiments of the disclosure.

FIG. 6B is a diagram of a system for preparing a wound treatment in the form of a skin graft material with a fat material according to embodiments of the disclosure.

FIG. 7 is a diagram of a method for preparing a wound treatment in the form of a skin graft material impregnated with a fat material according to embodiments of the disclosure.

FIG. 8 is a diagram of a system for preparing a wound treatment in the form of a skin graft material with a fat material according to embodiments of the disclosure.

FIG. 9A is a diagram of an embodiment of a system for preparing a wound treatment in the form of a skin graft material with a fat material according to embodiments of the disclosure.

FIG. 9B is a diagram of another embodiment of a system for preparing a wound treatment in the form of a skin graft material with a fat material according to embodiments of the disclosure.

FIG. 9C is a diagram of another embodiment of a system for preparing a wound treatment in the form of a skin graft material with a fat material according to embodiments of the disclosure.

FIG. 9D is a diagram of another embodiment of a system for preparing a wound treatment in the form of a skin graft material with a fat material according to embodiments of the disclosure.

FIG. 9E is a diagram of another embodiment of a system for preparing a wound treatment in the form of a skin graft material with a fat material according to embodiments of the disclosure.

FIG. 9F is a diagram of another embodiment of a system for preparing a wound treatment in the form of a skin graft material with a fat material according to embodiments of the disclosure.

FIG. 10 is a diagram of a system for preparing a wound treatment in the form of a skin graft material with a fat material according to embodiments of the disclosure.

FIG. 11 is a diagram of a method for preparing a wound treatment in the form of a skin graft material with a fat material according to embodiments of the disclosure.

FIG. 12 is a diagram of a system for preparing a wound treatment in the form of a skin graft material with a fat material according to embodiments of the disclosure.

FIG. 13 is a diagram of a system and a corresponding method for preparing a wound treatment in the form of a skin graft material with a fat material according to embodiments of the disclosure.

FIG. 14A is a hematoxylin and eosin stain image of a skin graft material that has not been subjected to annealing.

FIG. 14B is a hematoxylin and eosin stain image of a skin graft material that has not been subjected to annealing.

FIG. 15 is a diagram of a method for preparing a wound treatment including processing a skin graft material prior to application of a fat material according to embodiments of the disclosure.

FIG. 16A is a hematoxylin and eosin stain image of a skin graft material that has been subjected to annealing according to embodiments of the disclosure.

FIG. 16B is a hematoxylin and eosin stain image of a skin graft material that has been subjected to annealing according to embodiments of the disclosure.

FIG. 17 is a diagram of a method for preparing a wound treatment including processing a skin graft material prior to application of a fat material according to embodiments of the disclosure.

FIG. 18 is a diagram of another embodiment a system for preparing a wound treatment in the form of a skin graft material with a fat material according to embodiments of the disclosure.

### DETAILED DESCRIPTION

### Overview

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function, or “step for” performing a specific function is not to be interpreted as a “means” or “step” clause as specified in 35 U.S.C. § 112.

As used herein, the term “treatment” is intended to be understood by its common dictionary definition. That is, the term “treatment” broadly includes medical care and/or medicaments given to a patient for an illness or injury. A “treatment” may include the use of a chemical, physical, or biological agent to preserve or give particular properties to something. Thus, a “treatment” may be the medical care provided (i.e., in the form of a method or series of prescribed acts), or it may refer to the medicament used to preserve or give a particular property to something.

The term “wound” as used herein is intended to encompass tissue injuries generally. Thus, the term “wound” includes those injuries that cause, for example, cutting, tearing, and/or breaking of the skin such as lacerations, abrasions, incisions, punctures, avulsions, or other such injuries. Wounds may be described by any of the size, shape, or magnitude of the wound. For example, a paper cut is exemplary of a small, straight incision of relatively little magnitude, whereas a concussive blast resulting in a major laceration covering one or multiple body parts is exemplary of a relatively larger wound of greater magnitude. Each of the foregoing examples, however, fall within the scope of the term “wound,” as used herein.

The term “wound” additionally includes damage to underlying tissue, such as that caused by traumatic injury. As such, the term “wound” is intended to include a combination of multiple different wounds. For example, a traumatic amputation caused by an explosive blast may generally be referred to as a wound even though it is a compilation of a host of different lacerations, abrasions, avulsions, and punctures. Additionally, any underlying tissue damage resulting from the aforementioned explosive blast may further be encompassed within the understanding of this reference to a wound. The term “wound” is also intended to encompass tissue injuries caused by burns (e.g., thermal and/or chemical burns). Further, the term “wound” is also intended to encompass injuries resulting from, for example, diabetic foot ulcers, venous leg ulcers, surgical operations, pressure ulcers, and other causes.

Further, the wounds amenable to treatment by the wound treatment and methods disclosed herein include injuries that can be located in any site, including internal, interfacial, external, interstitial, extracorporeal, and/or intracorporeal. Examples of wounds suitable for coverage with the scaffold material include cuts, gashes, open wounds, tissue rupture, Decubitus, Dermatitis, lesions, chronic wounds, battlefield wounds, necrotic wounds, acute, chronic, traumatic, lacerations, abrasions, contusions, necrotizing fasciitis, toxic epidermal necrolysis, pressure wounds, venous insufficiency ulcers, arterial ulcers, diabetic or neuropathic ulcers, pressure ulcers, mixed ulcers, burn wounds, Mucormycosis, Vasculitic wounds, Pyoderma, gangrenosum, and equivalents, and/or combinations thereof. Treatment of wounds in human and animal subjects are contemplated.

A “traumatic wound,” as used herein refers to any wound resulting from physical injury that damages both the skin and underlying tissue. A gunshot wound is one non-limiting example of a traumatic wound, as it causes a puncture (i.e., a break) in the skin and ruptures or otherwise damages underlying tissue. As another non-limiting example, a concussive or explosive blast generally results in traumatic wound(s). Many, but not all, of the wounds received during wartime may be described as traumatic wounds due to the nature of war and war-related injuries. A “traumatic wound” can include hemorrhaging wounds, wounds with exposed bone and/or tendons, severe burns, deep tissue wounds (e.g., asymmetrical deep-tissue wounds), and/or large surface area wounds.

As described above, many different types of skin substitutes or skin graft materials may be used to aid in the healing process of a wound and to more quickly restore at least some of the functions of healthy skin. Skin substitutes may be considered broadly as a group of elements or materials that enable the temporary or permanent occlusion of a wound. Skin substitutes can generally be divided into biological skin substitutes, synthetic skin substitutes, or a hybrid skin substitute that includes biological and synthetic skin substitutes.

For the purposes of this application, the terms “skin substitutes,” “skin graft material,” “scaffold material,” “graft product,” or similar may include Kerecis™ Omega3 Wound by Kerecis, Kerecis™ Omega3 acellular fish skin from the Atlantic cod (Gadus morhua), or any other skin graft materials known for use in treating wounds or similar to the foregoing. The terms “extracellular matrix” or “ECM” as used herein refer to the non-cellular tissue material present within the fish skin that provides structural support to the skin cells in addition to performing various other important functions. The ECM described herein does not necessarily include matrix material that has been constituted or re-formed entirely from extracted, purified, or separated ECM components (e.g. collagen). But in some embodiments, an ECM used as a skin substitute may include matrix material that has been constituted or re-formed entirely from extracted, purified, or separated ECM components (e.g. collagen).

The extracellular matrix (ECM) of vertebrates is a complex structural entity surrounding and supporting cells. ECM is composed of complex mixtures of structural proteins, the most abundant of which is collagen, and other specialized proteins and proteoglycans. The scaffold material described herein is a largely intact acellular scaffold of natural biological ECM components from fish skin. The scaffold can also comprise naturally occurring lipids from the fish skin. The native three-dimensional structure, composition, and function of the dermal ECM is essentially unaltered, and provides a scaffold to support cell migration, adherence, proliferation, and differentiation, thus facilitating the repair and/or replacement of tissue.

The terms “acellular,” “decellularized,” “decellularized fish skin,” and the like as used herein refer to a biological skin material from which a substantial amount of cellular and nucleic acid content has been removed leaving a complex three-dimensional interstitial structure of ECM. In embodiments, “decellularized fish skin” may further entail fish skin which, in addition to the complex three-dimensional interstitial structure of ECM absent a substantial amount of cellular and nucleic acid content, includes omega 3 polyunsaturated fatty acids (PUFAs).

Other scaffold materials or skin graft materials may include those derived from mammalian skin/membranes, reconstituted from collagen materials, or artificially prepared. Generally, the scaffold material is from about 0.1 to 4.0 mm thick (i.e. in cross-section), such as 0.5, 1.0, 1.5, 2.0, 2.5, 3.0 or 3.5 mm thick. The thickness can depend on a number of factors, including the starting material used, processing, lyophilization, and/or rehydration. Of course, the thickness is proportionately greater when the product comprises more than one layer of scaffold material.

In embodiments, a scaffold material may be or comprise decellularized fish skin. The decellularized fish skin is configured to provide a scaffold material for supporting cell migration, adherence, proliferation, and differentiation for facilitating the repair and/or replacement of tissue, as described in U.S. Pat. No. 8,613,957, granted on Dec. 24, 2013, the application of which was filed Oct. 6, 2010, the contents of which are incorporated by reference herein in its entirety. The decellularized fish skin product described by U.S. Pat. No. 8,613,957 serves as a scaffold material that provides an intact scaffold for support for ingrowth of endothelial and/or epithelial cells. The decellularized fish skin scaffold material is biocompatible thus can be integrated by the host. Omega3 Wound is a commercially available skin substitute made from the minimally processed skin of wild caught Atlantic cod originating from Iceland. The fish skin is structurally alike to human skin with three basic layers including epidermis, dermis, and hypodermis and contains proteins, lipids, fatty acids, and other bioactive compounds that are homologous to human skin.

The decellularized fish skin can be particalized, comminuted, or otherwise processed into various sizes and shapes, as described in U.S. patent application Ser. No. 17/704,539, filed on Mar. 25, 2022, the contents of which are incorporated by reference herein in its entirety. The size of individual comminuted particles may vary, depending on the type and/or manner of comminution. For example, decellularized fish skin particles can be created through a jet milling process designed to output particles below a specified size. In some embodiments, decellularized fish skin is cut, chopped, or ground into particles, which may be done in a measured fashion to create uniform particles or roughly performed, thereby generating a variety of different sized particles.

A scaffold material in accordance with this invention may be obtained from intact fish skin. Any species of fish, including bony or cartilaginous fish, can be used as the source of the fish skin. For example, the source can be round fish like cod, haddock and catfish; flatfish, like halibut, plaice and sole; salmonids like salmon and trout; scombridaes like tuna; or small fish like herring, anchovies, mackerel and sardines. In certain embodiments the fish skin is obtained from cold-water oily fish and/or fish known to contain high amounts of omega-3 oil. Examples of fish high in omega-3 oil are salmon, pilchards, tuna, herring, cod, sardines, mackerel, sable fish, smelts, whitefish, hoki fish, and some varieties of trout.

The fish skin is removed from the fish before processing. If the fish skin is from a species of fish that has scales, the fish skin should be de-scaled so that a substantial portion of the scales are removed or at least the hydroxyapatite removed from the scales. The phrase “a substantial portion of the scales are removed” or “substantially scale-free” means that at least 95%, preferably at least 99%, and more preferably 100% of the scales on the fish skin are removed. “Substantially scale free” fish skin can also refer to fish skin from a fish species without scales. The scales are either removed prior to all processing, with purely mechanical pressure (via, e.g., knife, shaking with abrasives, water pressure, a special scale removal device that uses the same mechanical force as knives or other pressure device, like polishing with ceramic or plastic) or after some chemical treatment (e.g. decellularization) and then with mechanical pressure in order to wash the scales away. If the fish skin is first treated chemically and/or enzymatically (e.g. treatment with TRITON® X-100), the mechanical pressure generally needs to be gentle since the skin is more vulnerable to tearing after decellularization. The scales can be removed in more than one step, for example partial removal prior to decellularization followed by further removal during and/or after decellularization. Alternatively, the scales can be removed by chemical treatment alone.

After the scales have been removed, the fish skin is optionally frozen prior to decellularization. The fish skin can be frozen quickly by incubating the skin in liquid nitrogen or using other special freezing equipment that can freeze the skin to −70° C. or lower, in order to preserve the collagen structure of the scaffold. The freezing process may lyse or partially lyse the cells comprising the intact fish skin, and help facilitate decellularization of the fish skin. If the fish skin has been frozen, it can later be thawed for further processing.

Whether or not the fish skin was frozen, it can be washed with a buffer solution prior to further processing. For example, the fish skin can be washed 1-3 times with a buffer solution optionally containing one or more antioxidants (e.g. ascorbic acid (such as 50 mM ascorbic acid), Vitamins A, C, E, and beta carotene), antibiotics (e.g., streptomycin and penicillin), proteases (e.g. dispase II) and protease inhibitors (e.g. antipain, aprotinin, benzamidine, bestatin, DFP, EDTA, EGTA, leupeptin, pepstatin, phosphoramidon, and PMSF) to facilitate disinfection and stabilization of the fish skin. The buffer solution can be at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5. The buffer solution can also be used as a medium in which the fish skin can be stored for a few days up to a few weeks or more. In certain embodiments the fish skin is stored in the buffer solution at a temperature of about 4° C.

After freezing and/or washing and/or storage in a buffer solution, the fish skin is treated with one or more decellularizing solutions to remove cellular material, including antigenic material, from the fish skin with minimal to no damage to the mechanical and structural integrity and biological activity of the naturally occurring extracellular matrix.

“Decellularizing agents” are those agents that are effective in removing a substantial amount of cellular and nucleic acid content from the ECM. The ECM is “decellularized” or “substantially free” of cellular and nucleic acid content (i.e. a “substantial amount” has been removed) when at least 50% of the viable and non-viable nucleic acids and other cellular material have been removed from the ECM. In certain embodiments, about 55%, 60%, 65%, 70%, 75%, 80%, 85%, 90%, 91%, 92%, 93%, 94%, 95%, 96%, 97%, 98%, 99%, 99.5%, or 100% of the viable and non-viable nucleic acids and cellular material are removed. Decellularization can be verified by, for example, testing the treated fish skin for DNA content. Removal of the nucleic acids from the ECM can be determined, for example, by histological examination of the ECM, and/or by a biochemical assay such as the PICOGREEN® assay, diphenylamine assay, or by PCR.

Decellularization disrupts the cell membranes and releases cellular content. Decellularizing may involve one or more physical treatments, one or more chemical treatments, one or more enzymatic treatments, or any combination thereof. Examples of physical treatments are sonication, mechanical agitation, mechanical massage, mechanical pressure, and freeze/thawing. Examples of chemical decellularizing agents are ionic salts (e.g. sodium azide), bases, acids, detergents (e.g. non-ionic and ionic detergents), oxidizing agents (e.g. hydrogen peroxide and peroxy acids), hypotonic solutions, hypertonic solutions, chelating agents (e.g. EDTA and EGTA), organic solvents (e.g. tri(n-butyl)-phosphate), ascorbic acid, methionine, cysteine, maleic acid, and polymers that bind to DNA (e.g. Poly-L-lysine, polyethylimine (PEI), and polyamindoamine (PAMAM)). Non-ionic detergents include 4-(1,1,3,3-Tetramethylbutyl)phenyl-polyethylene glycol, t-Octylphenoxypolyethoxyethanol, Polyethylene glycol tert-octylphenyl ether (TRITON® X-100) (Dow Chemical Co.). Ionic detergents include sodium dodecyl sulfate (SDS), sodium deoxycholate, TRITON® X-200, and zwitterionic detergents (e.g. CHAPS). Other suitable decellularizing detergents include polyoxyethylene (20) sorbitan mono-oleate and polyoxyethylene (80) sorbitan mono-oleate (Tween 20 and 80), 3-[(3-chloramidopropyl)-dimethylammino]-1-propane-sulfonate, octyl-glucoside and sodium dodecyl sulfate. Examples of enzymatic decellularizing agents are proteases, endonucleases, and exonucleases. Proteases include serine proteases (e.g. trypsin), threonine proteases, cysteine proteases, aspartate proteases, metalloproteases (e.g. thermolysin), and glutamic acid proteases. Decellularization is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

An example of a decellularization step is incubating the fish skin in a solution comprising 1 M NaCl, 2% deoxycholic acid, 0.02% sodium azide and 500 ppm streptomycin. In another example, the fish skin is incubated with a first decellularizing solution comprising a protease (e.g., 2.5 U/mL dispase II) and other components (e.g., 0.02% sodium azide). The first decellularizing solution is poured off and the fish skin is then treated with a second decellularizing solution such as a solution comprising a detergent (e.g., 0.5% TRITON® X-100) and other components (e.g. 0.02% sodium azide). In another example, the fish skin is first treated with a decellularizing solution comprising detergent (e.g. 0.5% TRITON® X-100) with other components (e.g. 0.02% EDTA, sodium azide, and/or deoxiholic acid), and then incubated in a second decellularizing solution comprising a detergent such as SDS.

The decellularizing step(s) can be repeated as needed by pouring off any remaining decellularizing solution, optionally washing the fish skin with a buffer solution (e.g. Hank's Balanced Salt Solution), and then treating the fish skin again with another step of decellularization. Once a sufficient amount of cell material has been removed, the decellularizing solution can be removed (e.g., by aspiration or by gently pouring out the solution).

After decellularization, the fish skin can optionally be washed with water, buffer solution, and/or salt solution. Examples of suitable washing solutions include Dulbecco's phosphate buffered saline (DPBS), Hank's balanced salt solution (HBSS), Medium 199 (M199, SAFC Biosciences, Inc.) and/or L-glutamine. Washing step(s) are generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The fish skin can optionally be bleached to improve the appearance of the final product. Bleaching can be carried out before, after, and/or concurrently with decellularization. For example, one or more bleaching agent can be incorporated into one or more of the decellularization solution(s) and/or into one or more buffer solution(s). Examples of bleaching agents include sodium sulfite, hydrogen peroxide, ammonium persulfate, potassium persulfate, and sodium persulfate. In certain embodiments, if a strong bleaching agent like persulfate(s) are used, bleaching and decellularization can be combined in a single step comprising incubating the fish skin in a mixture of one or more bleaching agents, thickeners, and peroxide sources. For example, a dry bleaching mixture can be prepared (see, e.g., the “bleaching mixtures” described in Example 5), followed by the addition of water, hydrogen peroxide, or a combination thereof to the dry mixture to form a bleaching solution that may also be sufficient for decellularization. The bleaching agents (e.g. sodium sulfite, hydrogen peroxide, ammonium persulfate, potassium persulfate, and sodium persulfate) should be about 40-60% w/w of the dry mixture. A combination of EDTA and persulfates may be added to the mixture to accelerate bleaching as well as decellularization.

In certain embodiments the concentration of EDTA in the dry mixture is about 0.25-5% w/w. Hydrogen peroxide can be about 15-25% of the mixture; the peroxide source can be sodium percarbonate and potassium percarbonate. Sodium phosphate perhydrate and sodium carbonate or magnesium metasilicate and silicium silicate can also be used as a peroxide source. The dry mixture can also include silica and hydrated silica, at for example 1-10% w/w, and optionally one or more stearate (e.g. ammonium stearate, sodium stearate, and/or magnesium stearate). In addition the dry mixture can optionally include thickeners, such as hydroxypropyl methylcellulose, hydroxyethylcellulose, algin (i.e. alginate), organic gums (e.g. cellulose, xanthan gum) sodium metasilicate, and combinations thereof to increase the viscosity of the bleaching/decellularization solution and protect protein fibers from damage. Bleaching, and/or bleaching plus decellularization, is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0,e.g. between 7.5 and 8.5. After bleaching and/or bleaching plus decellularization, the fish skin is optionally washed with a solution comprising L-glutamine under the pH conditions described above.

In certain embodiments, the fish skin is treated with a digestion enzyme. Similar to bleaching, digestion can be carried out before, after, and/or concurrently with decellularization. Suitable enzymes include proteases, for example serine proteases, threonine proteases, cysteine proteases, aspartate proteases, metalloproteases, and glutamic acid proteases. In certain embodiments the digestion enzyme is a serine protease such as trypsin. The digestion enzyme can be an enzyme that functions in an alkaline environment, limits cross-linking within the ECM, and softens the fish skin. Digestion is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The decellularized fish skin can optionally be cryopreserved. Cryopreservation can involve immersing the fish skin in a cryoprotectant solution prior to freezing. The cryoprotectant solution generally comprises an appropriate buffer, one or more cryoprotectants, and optionally a solvent, e.g. an organic solvent which in combination with water undergoes minimal expansion and contraction. Examples of cryoprotectants include sucrose, raffinose, dextran, trehalose, dimethylacetamide, eimethylsulfoxide, ethylene glycol, glycerol, propylene glycol, 2-Methyl-2.4-pantandial, certain antifreeze proteins and peptides, and combinations thereof. Alternatively, if the decellularized fish skin is fast-frozen (flash-frozen) prior to sublimation in order to minimize ice crystals formed during the freezing step, the skins can optionally be frozen in a buffer solution that does not include cryoprotectants. Cryopreservation is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The decellularized fish skin can be packaged inside a sterile container, such as a glass vial or a pouch. In one embodiment, a TYVEK® pouch is used. For example, the fish skin can be incubated in a cryoprotectant solution, packaged in a TYVEK® pouch and then placed into a freeze dryer and frozen at a rate which is compatible with the cryoprotectant.

The decellularized fish skin can be lyophilized, i.e. frozen at a low temperature and under vacuum conditions so that water is removed sequentially from each ice crystal phase without ice re-crystallization. During lyophilization, water is generally removed first via sublimation and then via desorption if necessary. Another method of removing excess water after processing and before sterilization is vacuum pressing.

In certain embodiments, the decellularized fish skin is sterilized before and/or after being frozen. Sterilization methods are well known in the art. For example, the decellularized fish skin can be placed in an ethylene oxide chamber and treated with suitable cycles of ethylene oxide. Other sterilization methods include sterilizing with ozone, carbon dioxide, gaseous formaldehyde or radiation (e.g. gamma radiation, X-rays, electron beam processing, and subatomic particles).

As an alternative to or in addition to freezing, freeze-drying and/or vacuum pressing of water, the decellularized fish skin can be preserved in a non-aqueous solution such as alcohol.

The resulting product (scaffold material) is a sterile, collagen-based matrix that possesses properties that may facilitate the regeneration, repair and/or replacement of tissue (e.g., repair, regeneration, and/or growth of endogenous tissue). The term “scaffold material” may refer in certain embodiments to material comprising fish skin that has been decellularized and optionally bleached, digested, lyophilized, etc. as discussed above. In varying aspects, the term “scaffold material” may refer to graft products, such as decellularized fish skin, that has been particularized, i.e. shredded.

The scaffold material can provide an intact scaffold for support of endothelial and/or epithelial cells, can be integrated by the host, is biocompatible, does not significantly calcify, and can be stored and transported at ambient temperatures. The phrase “integrated by the host” means herein that the cells and tissues of the patient being treated with the scaffold material can grow into the scaffold material and that the scaffold material is actually integrated/absorbed into the body of the patient. The term “biocompatible” refers to a material that is substantially non-toxic in the in vivo environment of its intended use, and that is not substantially rejected by the patient's physiological system (i.e., is non-antigenic).

This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part 1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The scaffold material may contain proteins from the ECM of the fish skin. The ECM components in the scaffold material can include, for example, structural proteins; adhesive glycoproteins; proteoglycans; non-proteoglycan polysaccharides; and matricellular proteins. Examples of structural proteins include collagens (the most abundant protein in the ECM), such as fibrillar collagens (types I, II, III, V, and XI); facit collagens (types IX, XII, and XIV), short chain collagens (types VIII and X), basement membrane collagen (type IV), and other collagens (types VI, VII, and XIII); elastin; and laminin. Examples of adhesive glycoproteins include fibronectin; tenascins; and thrombospondin. Examples of proteoglycans include heparin sulfate; chondroitin sulfate; and keratan sulfate. An example of a non-proteoglycan polysaccharide is hyaluronic acid. Matricellular proteins are a structurally diverse group of extracellular proteins that regulate cell function via interactions with cell-surface receptors, cytokines, growth factors, proteases, and the ECM. Examples include thrombospondins (TSPs) 1 and 2; tenascins; and SPARC (secreted protein, acidic and rich in cysteine).

In certain embodiments, decellularization (and other optional processing steps) does not remove all of the naturally occurring lipids from the lipid layer of the fish skin. Thus, the scaffold material can comprise one or more lipids from the fish skin, particularly from the lipid layer of the fish skin. For example, the scaffold material may include up to about 25% w/w lipids (of dry weight of the total scaffold material after lyophilization), such as 0.1%, 0.5%, 1%, 2%, 3%, 4%, 5%, 6% 7%, 8%, 9%, 10%, 11%, 12%, 13%, 14%, 15%, 16%, 17%, 18%, 19%, 20%, 21%, 22%, 23%, or 24% w/w lipids. The presence of lipids in the fish skin can be verified, for example, by organic solvent extraction followed by chromatography. Examples of suitable organic solvents include acetone and chloroform.

The lipids in the fish skin can include, for example, fatty acyls (i.e. fatty acids, their conjugates, and derivates); glycerolipids; glycerophospholipids (i.e. phospholipids); sphingolipids; saccharolipids; polyketides; sterol lipids (i.e. sterols); certain fat-soluble vitamins; prenol lipids; and/or polyketides. Examples of fatty acyls include saturated fatty acids, such as polyunsaturated fatty acids; fatty esters; fatty amides; and eicosanoids. In certain embodiments the fatty acids include omega-3 fatty acids, such as eicosapentaenoic acid (EPA) and docosahexaenoic acid (DHA) (found in high concentration in fish oil). Other fatty acids found in fish oil include arachidic acid, gadoleic acid, arachidonic acid, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, and lignoceric acid. Examples of glycerolipids include mono-, di-, and tri-substituted glycerols, such as monoacylglycerols, diacylglycerols, and triacylglycerols (i.e. monoglycerides, diglycerides, and triglycerides). Examples of glycerophospholipids include phosphatidylcholine; phosphatidylethanolamine; and phosphatidylserine. Examples of sphingolipids include phosphosphingolipids and glycosphingolipids. Examples of sterol lipids include cholesterol; steroids; and secosteroids (various forms of Vitamin D). Examples of prenol lipids include isoprenoids; carotenoids; and quinones and hydroquinones, such as Vitamins E and K.

The scaffold material can be used in dried form. Alternatively, the scaffold material can be rehydrated prior to use. In certain embodiments, one or more scaffold materials are laminated together to form a thicker scaffold material.

A scaffold material may be provided in the form of particles in embodiments, each of the particles configured for actively promoting wound healing as a physical scaffold for infiltrating cells involved in wound healing/repair, such as for cell ingrowth and neovascularization. Scaffold material particles of embodiments may be configured to advantageously retain the three-dimensional (“3D”) structure of the scaffold material, for example with an ECM that is recognizable, for instance, on a histology analysis. The dimensions of the particles may additionally be configured so as to facilitate molding, packing, or otherwise applying the particles into a wound cavity with greater precision than existing approaches to wound therapy.

In embodiments, the scaffold material particles have a greatest dimension within a predetermined maximum size threshold and a minimum size threshold that is effective to preserve the matrix structure of the decellularized fish skin and to promote cellular regenerative ingrowth into a wound. That is, a greatest dimension, such as a greatest one of a length, width, and/or thickness of the particles, may be lower than a maximum size, such as 1 mm, and larger than a minimum size, such as a size at which the ECM is destroyed. In embodiments, shredded, decellularized fish skin particles are obtained for a scaffold material by providing a sheet of decellularized fish skin as described above and then shredding the sheet of decellularized fish skin and optionally sieving the shredded particles until the shredded, decellularized fish skin particles are within the predetermined minimum and maximum size thresholds.

Biological skin substitutes may include, but are not limited to, skin grafts, including autologous skin grafts, syngeneic skin grafts, allogeneic skin grafts, xenogeneic skin grafts such as porcine skin grafts, cadaveric skin allografts, and amniotic tissue grafts. Certain biological skin substitutes may be made from biologic materials containing intact collagen or reconstituted collagens. Examples include brands such as: Oasis, Matristem, Integra and Puracol. Those products are often referred to by clinicians as being matrix products. The matrix products are inserted into the wound where they are to attract cellular ingrowth. A secondary wet-to-dry wound dressing may then be applied on top of the wound dressing. The decellularized fish skin described in U.S. Pat. No. 8,613,957, is one example of a matrix product. The decellularized fish skin product described by U.S. Pat. No. 8,613,957 serves as a scaffold material that provides an intact scaffold for support for ingrowth of endothelial and/or epithelial cells.

Examples of other biological skin substitutes include those described in U.S. Pat. No. 6,541,023, which describes the use of porous collagen gels derived from fish skin for use as tissue engineering scaffolds. Preparation of the collagen gels involves grinding the fish skin. Additionally, Chinese Patent No. 1068703 describes a process for preparing fish skin for dressing burn wounds, involving separating fish skin from the fish body and placing the skin in a preservation solution of iodine tincture, ethanol, borneol, sulfadiazine zinc and hydrochloric acid in amounts sufficient to establish a pH value of 2.5-3. However, these products can be difficult to handle as the product of U.S. Pat. No. 6,541,023 is in a gel form and the product of China Patent No. 1068703 is stored in a solution.

In addition, a number of extracellular matrix products for medical uses have been derived from human skin (ALLODERM® Regenerative Tissue Matrix (LifeCell)); fetal bovine dermis (PRIMATRIX™ Dermal Repair Scaffold (TEI Biosciences)); porcine urinary bladder (MATRISTEM™ Extracellular Matrix Wound Sheet (Medline Industries, Inc.)); and porcine small intestinal submucosa (OASIS® Wound Matrix (Healthpoint Ltd.)).

Examples of commercially available synthetic skin substitutes include Biobrane®, Dermagraft®, Integra®, Apligraf®, MatriDerm®, OrCel®, Hyalomatrix®, and Renoskin®.

U.S. Published Patent Application No. 2003/0059460 discloses a hybrid polymer skin substitute material comprising synthetic and natural polymers that can be used in regenerating living body tissue. The hybrid comprises a cross-linked naturally-occurring polymer and a biodegradation-absorbable synthetic polymer. A series of complicated process steps, however, must be undertaken to produce the hybrid material. In addition, the resulting hybrid material contains synthetic as well as naturally-occurring materials.

As noted above, the group of skin substitutes which can be used as examples of skin substitutes according to the present disclosure is large and varied. The AHRQ Technology Assessment Program entitled “Skin Substitutes for Treating Chronic Wounds” Technical Brief Project ID WNDT0818, published Feb. 2, 2020, which is incorporated herein by reference, in Table 2, on pages 9-13, identified 76 commercially available products with few studies comparing them internally. Each of these listed skin substitutes may be an embodiment of a skin substitute according to the present disclosure.

In various examples, a coloring agent may be contemplated for use with a skin graft material, as described in U.S. patent application Ser. No. 17/703,650, filed on Mar. 24, 2022, the contents of which are incorporated by reference herein in its entirety. In its broadest sense, the coloring agent contemplated herein is a coloring agent, or combination of color or coloring agents, that provides a color to the skin substitute that changes or loses color, based on changes of conditions within the wound during the healing process or changes to the skin substitute. In preferred embodiments, the coloring agent may degrade upon attack by one or more proteases within the wound. With such a coloring agent, the color agent, upon degradation by the one or more proteases, loses its color. For example, the coloring agent may provide a blue or violet color to the skin substitute. But upon attack by one or more proteases within the wound after application of the wound treatment including the skin substitute and the coloring agent, the color of skin substitute of the wound treatment is also degraded or lost, whereby the color of the applied wound treatment changes to the original color of the skin substitute or to a different color. But the change of color of the coloring agent is not limited thereto, and may include a color shift upon a change of condition within the wound. For example, the color provided by the coloring agent may be triggered such that the original color of the skin substitute is not changed upon application or adding of the coloring agent. But a color change of the coloring agent may be triggered or caused by a change of conditions within the wound, thereby turning the skin substitute of the wound treatment to a new or different color than the original color of the skin substitute.

For the purposes of this application, the terms “fat material,” “subcutaneous fat,” “dermal fat material,” “adipose tissue,” or “fat graft” may include or otherwise derive from biological components of fat. In varying examples, a fat material may include a lipoaspirate material, such as resulting from liposuction or similar procedures. A fat material may include one or more of adipocytes, endothelial cells, fibroblasts, B and T-lymphocytes, macrophages, myeloid cells, pericytes, pre-adipocytes, smooth muscle cells, collagens, fibronectin, laminin, and stromal cells. The fat material may further include blood, lipids, debris from adipocyte rupture, and/or any surrounding biological components. It should be noted that visceral fat material is not excluded from the disclosed embodiments, although embodiments may be described with respect to subcutaneous fat material for convenience and to reflect more common use.

As noted above, a fat material may comprise a lipoaspirate obtained via a liposuction procedure. Generally, liposuction refers to a surgical procedure involving the use of suction, such as through a cannula, to remove fat material from a portion of the body. A liposuction procedure may include any known procedure for removal of fat material, such as tumescent liposuction, dry liposuction, wet liposuction, etc. In varying aspects, a fat material may comprise apical fat, mantle fat, deep fat, camper's fascia, sub-scarpa's fascia, and/or another fat layer material. A fat material may be obtained from any portion of a body having fat material and related tissue.

In embodiments, a fat material may be recently harvested or may have been preserved for an extended period prior to use. A fat material may be subjected to processing following extraction or may be provided directly to a skin graft material according to varying embodiments. In wound treatments according to the present disclosure, a skin graft material may be prepared for use on a patient by application of a fat material harvested from the same patient, another patient, lab grown material, and/or an animal. Advantageously, the use of fat material harvested from the patient having the wound for treatment allows for improved healing, allows for a ready source of fat material, and prevents allergenic or similar complications.

### Various Embodiments and Components for Use Therewith

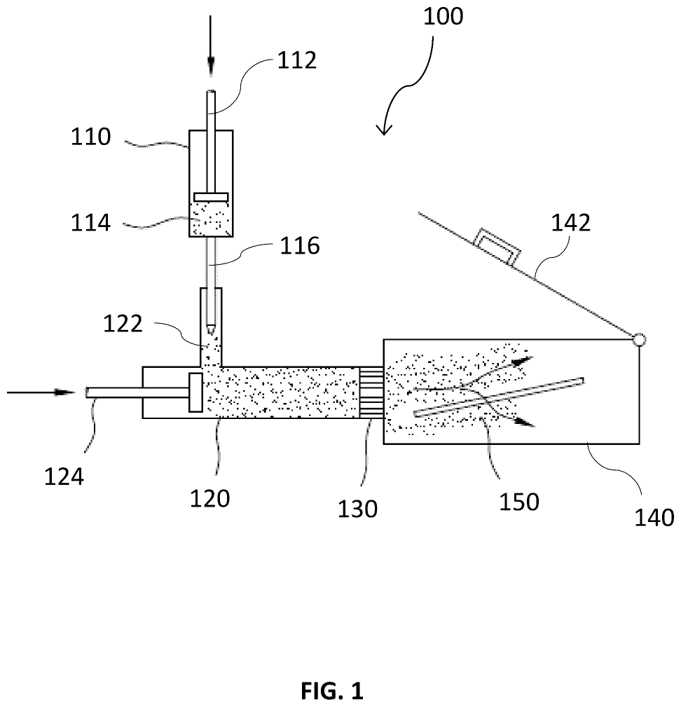

FIG. 1 illustrates a system 100 for preparing a wound treatment according to an embodiment of the present disclosure. The system 100 may include a syringe 110 or similar device for providing a fat material to a loading chamber 120. The loading chamber 120 may be connected by an extrusion die 130 to a preparation chamber 140, the preparation chamber 140 configured to retain a skin graft material 150 therein.

In one aspect, the system 100 may be provided as a kit or device for preparing a wound treatment for application to a wound of a patient. In this respect, a skin graft material 150 may be loaded into the preparation chamber 140 via a closing element 142 in the form of a door, flap, or similarly accessible portion of the preparation chamber 140. The skin graft material 150 may be retained in a predetermined position in the preparation chamber 140 by the use of a stage, clips, the closing element 142, clamps, or the like.

A fat material may be obtained from the patient or another source via the syringe 110 or similar device, such as via a liposuction procedure. In the embodiment of FIG. 1, the syringe 110 includes a syringe plunger 112 for generating positive or negative pressure in a syringe body 114, in order to load or unload a fat material therein via a cannula 116 or needle. The syringe 110 may be configured to cooperate with an input channel 122 of the loading chamber 120 for loading the fat material into the loading chamber 120. For example, the input channel 122 may have a length, diameter, or another dimension configured to match a length, diameter, or another dimension of the cannula 116. In certain aspects the input channel 122 may have an inner diameter of 6 mm or less, more particularly 4 mm or less. In embodiments, the input channel 122 may include an elastic material configured to elastically contract against the cannula 116, such that an inner diameter of the input channel 122 corresponds to an outer diameter of the cannula 116. Matching an input channel 122 and a cannula 116 according to embodiments of the current disclosure may advantageously prevent any fat material from escaping the loading chamber 120 via the input channel 122.

The loading chamber 120 may form an extrusion device by use of a plunger 124 or similar pressure generating device configured to force a fat material out of the loading chamber 120 through the extrusion die 130. In embodiments, the plunger 124 may be configured to apply and/or generate a pressure in the loading chamber 120 from the application of a manual force, such that extrusion of the fat material may be caused by hand. The extrusion die 130 may be configured to shape the fat material into smaller portions for application to the skin graft material 150 in the preparation chamber 140. In certain aspects of the disclosed embodiments, application of the fat material to the skin graft material 150 may comprise infusing, impregnating, mixing or otherwise joining the fat material and the skin graft material 150. In varying embodiments, the preparation chamber 140 may be configured to retain the skin graft material 150 directly against the extrusion die 130 or may be configured to hold the skin graft material 150 at another predetermined position for receiving the extruded fat material.

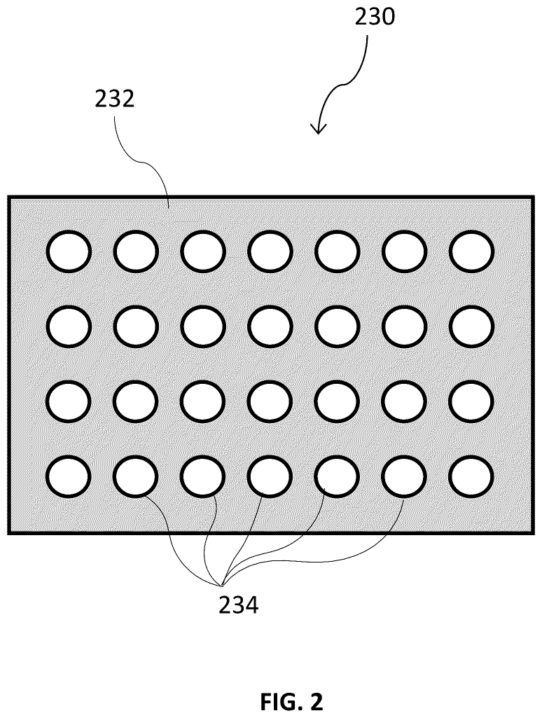

Referring to FIG. 2, an extrusion die 230 according to embodiments of the disclosure may comprise a face 232 defining one or more openings 234 therethrough. The one or more openings 234 may be configured to have a circular shape, a square shape, a rectangular shape, or any other shape. The one or more openings may have a minimum dimension perpendicular to the movement of the fat material of 2 mm or less, 1 mm or less, or 0.5 mm or less, such that individual components of the fat material are separated and/or reduced in size for application to a scaffold material. To facilitate separation of individual components of the fat material, the one or more openings 234 may be provided with sharpened edges and/or blades in the one or more openings 234, the sharpened edges or blades facing the plunger 124.

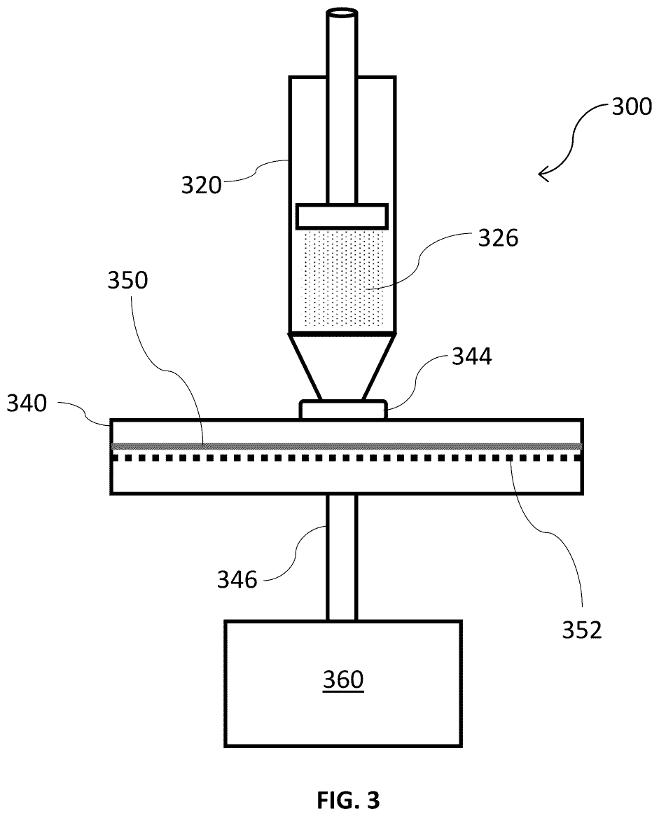

In another aspect, a system 300 for preparing a wound treatment may be provided according to FIG. 3. In the system 300, a loading chamber 320 containing a fat material 326 may be connected to a preparation chamber 340 retaining a skin graft material 350 therein. The preparation chamber 340 may include an input channel 344 configured to cooperate with the loading chamber 320. In certain examples, the loading chamber 320 may comprise a syringe or syringe-like device. The input channel 344 may be configured to detachably secure to the loading chamber 320 or may otherwise have a shape corresponding to an end of the loading chamber 320, advantageously facilitating a transfer of the fat material 326 from the loading chamber 320 to the preparation chamber 340. In embodiments, the input channel 340 may be configured to open and close, such as by applying a sealing element thereto or actuating a valve therein.

A screen mesh 352 may be provided in the preparation chamber 340 between the input channel 344 and an output channel 346. The screen mesh 352 may be configured to retain the skin graft material 350 in the preparation chamber 340 against a positive or negative pressure therein, while allowing the fat material 326 to pass through. In embodiments, a vacuum pump 360 may be connected to the output channel 346 for generating a negative pressure in the preparation chamber 340. The vacuum pump 360 may pull the fat material 326 from the loading chamber 320, into the preparation chamber 340 and through both the skin graft material 350 and the screen mesh 352. In this manner, the fat material 326 may be applied to the skin graft material 350, preparing a wound treatment according to the present disclosure.

In some embodiments, the vacuum pump 360 may comprise a peristaltic pump, a syringe or similar device. The loading chamber 320 may further be configured to force the fat material 326 into the preparation chamber 340 under a compressing force, such as by actuation of a plunger or similar element as described with respect to the system 100 of FIG. 1. In varying aspects, the input channel 344 may be provided with an extrusion die, blades or the like for separating individual components or portions of the fat material 326 as the fat material 326 is passed to the impregnating chamber 340.

In some embodiments, the screen mesh 352 may have a screen size of 3 mm or less, 2 mm or less, or 1 mm or less. The screen mesh 352 may be positioned between the skin graft material 350 and the output channel 346, such as to prevent the skin graft material 350 from contacting or entering into the output channel 346. In certain embodiments, the screen mesh 352 may be secured to the preparation chamber 340 or may have a size and shape corresponding to a size and shape of the preparation chamber 340, such that the screen mesh 352 is substantially retained in a predetermined position therein. In one aspect, a further screen mesh 352 may be provided between the skin graft material 350 and the input channel 344, such that the further skin graft material 352 may separate individual components or portions of the fat material 326 as the fat material 326 is passed through the preparation chamber 340.

The preparation chamber 340 may comprise a flexible bag or rigid chamber, defining an interior volume accessible via a closing element such as a door, flap, or similarly accessible portion of the preparation chamber 340. As in the system 100 of FIG. 1, the skin graft material 350 may be retained in a predetermined position in the preparation chamber 340 using a stage, clips, the closing element, clamps, or the like.

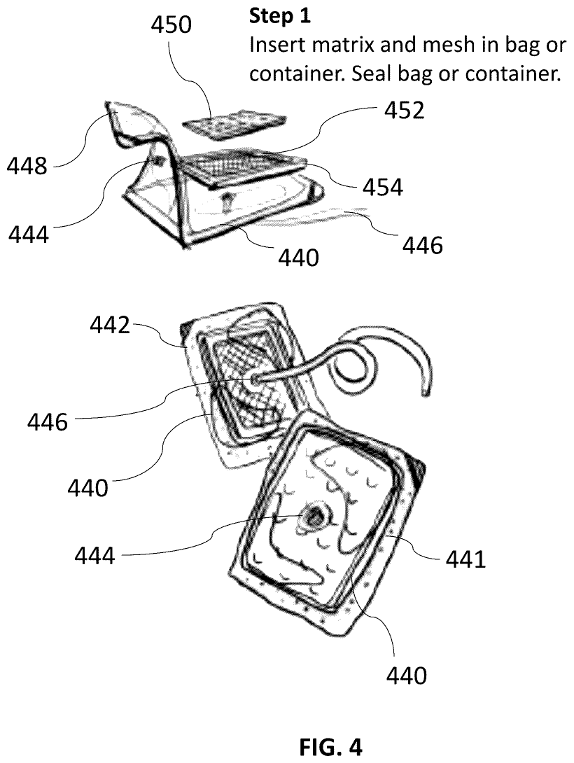

FIG. 4 illustrates an embodiment of the preparation chamber 440 comprising a flexible bag including a sealable flap 448. The sealable flap 448 may include an adhesive or similar connecting means for enclosing and/or sealing the preparation chamber 440. As shown, a screen mesh 452 and a skin graft material 450 may be provided to the preparation chamber 440 and enclosed therein using the sealable flap 448. In certain aspects, the screen mesh 452 may be configured as a tray for receiving the skin graft material 450, such as having a ridge element 454 defining an outer periphery thereof. An input channel 444 may be provided in a first side 441 of the preparation chamber 440 opposite an output channel 446 in a second side 443 of the preparation chamber 440.

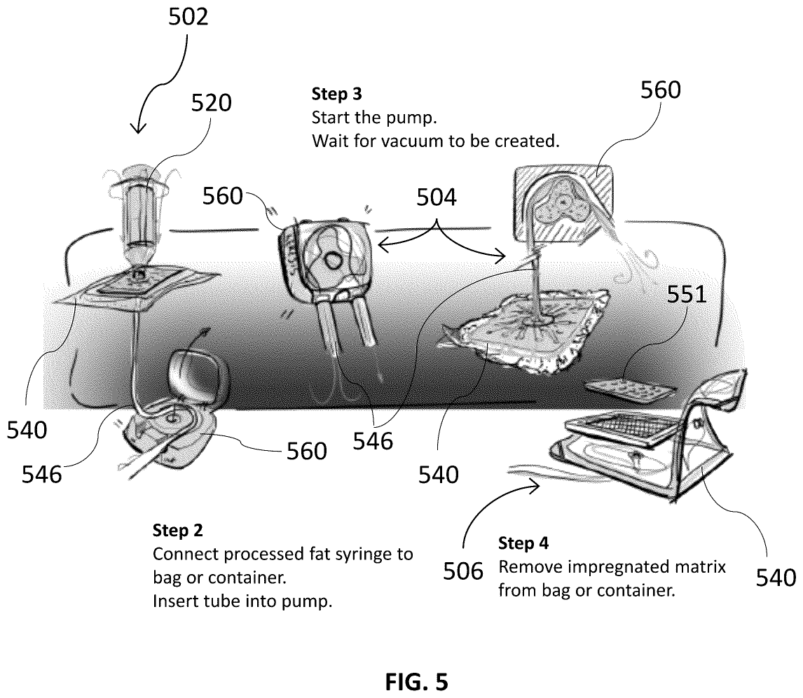

Operation of a system 300 for preparing a wound treatment may be better understood with reference to the flow diagram of FIG. 5. As illustrated in the embodiment of FIG. 5, a loading chamber 520 may be provided in the form of a syringe loaded with a fat material. In a first step 502, the loading chamber 520 may be connected to a preparation chamber 540 including a skin graft material and a screen mesh therein, for example according to the embodiment of FIG. 4. A vacuum pump 560 in the form of a peristaltic pump may be connected to the preparation chamber 540 via an outlet channel 546 in the form of a tube, such as in the form of surgical tubing connected to the preparation chamber 540.

The vacuum pump 560 may be activated in a second step 504 to generate a vacuum pressure in the preparation chamber 540 via the outlet channel 546. The vacuum pressure may draw the fat material from the loading chamber 520 into the preparation chamber 540 and through both the skin graft material and the screen mesh, such that the fat material may be applied to the skin graft material. In varying aspects, the outlet channel 546 may be configured to eject any fat material drawn therethrough or may be configured to collect the fat material, whether for another use or for disposal. When a desired level of application of the fat material is achieved in the skin graft material 550, the preparation chamber 540 may be opened and the skin graft material with applied fat material 551 may be removed in step 506. The prepared skin graft material 551 may then be used as a wound treatment, such as by applying the same to a wound, or may be stored for use on a wound within a predetermined period.

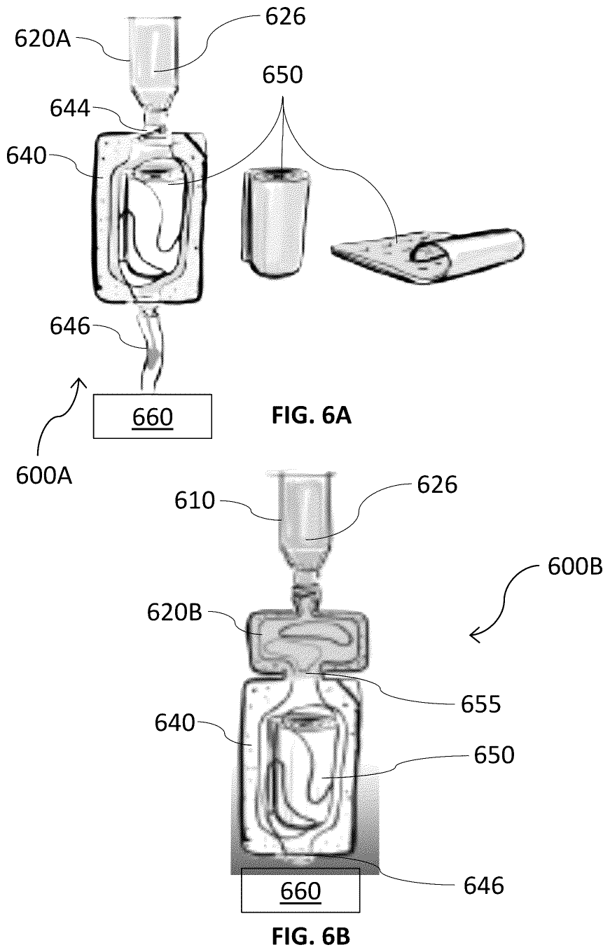

In embodiments according to FIGS. 6A and 6B, a system 600A, 600B may be provided for preparing a wound treatment using a non-planar skin graft material, an agglomeration of comminuted or particalized skin graft material, or using a skin graft material 650 provided in a rolled or folded configuration. Similar in some respects to the embodiment of FIG. 3, in the system 600A a loading chamber 620A containing a fat material 626 may be connected to a preparation chamber 640 retaining a skin graft material 650 therein. The preparation chamber 640 may include an input channel 644 configured to cooperate with the loading chamber 620A. In certain examples, the loading chamber 620A may comprise a syringe or syringe-like device.

Similar in some respects to the embodiment of FIG. 1, the system 600B may include a syringe 610 or similar device for providing a fat material to a loading chamber 620B. The loading chamber 620B containing a fat material 626 may be connected to a preparation chamber 640 retaining a skin graft material 650 therein. The preparation chamber 640 may include an input channel 655 configured to cooperate with the loading chamber 620B. In certain examples, the loading chamber 620B may comprise a sealable compartment that may receive a fat material from the syringe 610.

A vacuum pump 660 or similar vacuum source may be connected to an output channel 646 of the preparation chamber 640 for generating a negative pressure in the preparation chamber 640. The vacuum pump 660 may pull the fat material 626 from the loading chamber 620A, 620B, into the preparation chamber 640 and through the non-planar, comminuted, or rolled skin graft material 650. In certain embodiments, dimensions of the input channel 655 and/or the output channel 646 may be configured to prevent the skin graft material 650 from being drawn or forced out of the impregnating chamber 640, such that the fat material 626 may be applied to the skin graft material 650. In certain embodiments, the vacuum pump 660 may comprise a syringe, for example the same syringe 610 used to load the loading chamber or another syringe.

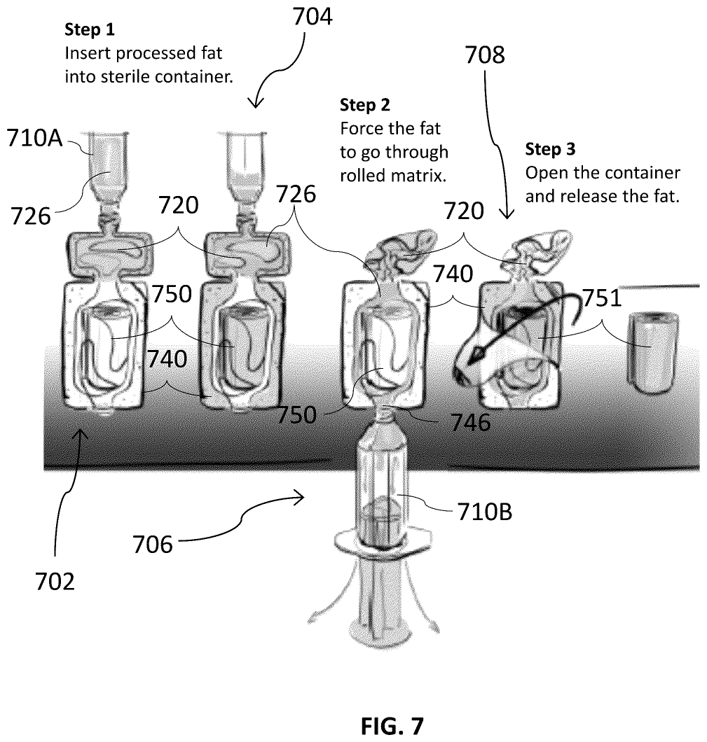

Operation of a system 600A, 600B for preparing a wound treatment may be better understood with reference to the flow diagram of FIG. 7. As illustrated in the embodiment of FIG. 7, a syringe 710A loaded with a fat material 726 may be provided to a loading chamber 720 in a first step 702. In a second step 704, the loading chamber 720 may be loaded with the fat material 726 from the syringe 710A, such as by expelling the syringe 710 contents via a plunger of the syringe 710A. In a third step 706, a syringe 710B, whether the same syringe or another syringe, may be connected to a preparation chamber 740 including a skin graft material 750 therein, for example a rolled skin graft material. The syringe 710B may be connected to the preparation chamber 740 via an outlet channel 746 opposite the loading chamber 720 and may be used to generate a negative pressure in the preparation chamber 740 during the third step 706.

The negative pressure may draw the fat material 726 from the loading chamber 720 into the preparation chamber 740 and through the skin graft material 750, such that the fat material 726 may be applied to the skin graft material 750. When a desired level of application of the fat material is achieved in the scaffold material 750, the preparation chamber 740 may be opened and the skin graft material with applied fat material 751 may be removed in step 708. The prepared skin graft material 751 may then be used as a wound treatment, such as by applying the same to a wound, or may be stored for use on a wound within a predetermined period.

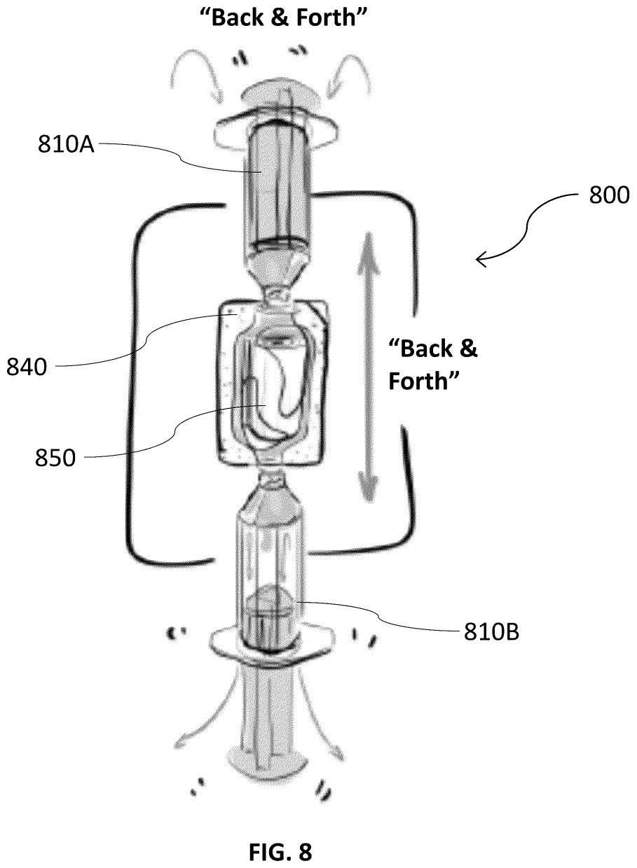

In varying embodiments, a system 800 may be configured to move a fat material across a skin graft material 850 contained in a preparation chamber 840 in a repeated manner, such as by drawing the fat material back and forth between two syringes 810A, 810B or vacuum pumps according to FIG. 8. By repeated circulation through the skin graft material 850, application of the fat material to the skin graft material 850 may be more completely achieved without an increase in the amount of fat material required, for example advantageously reducing the lipoaspirate that may need to be removed from the patient.

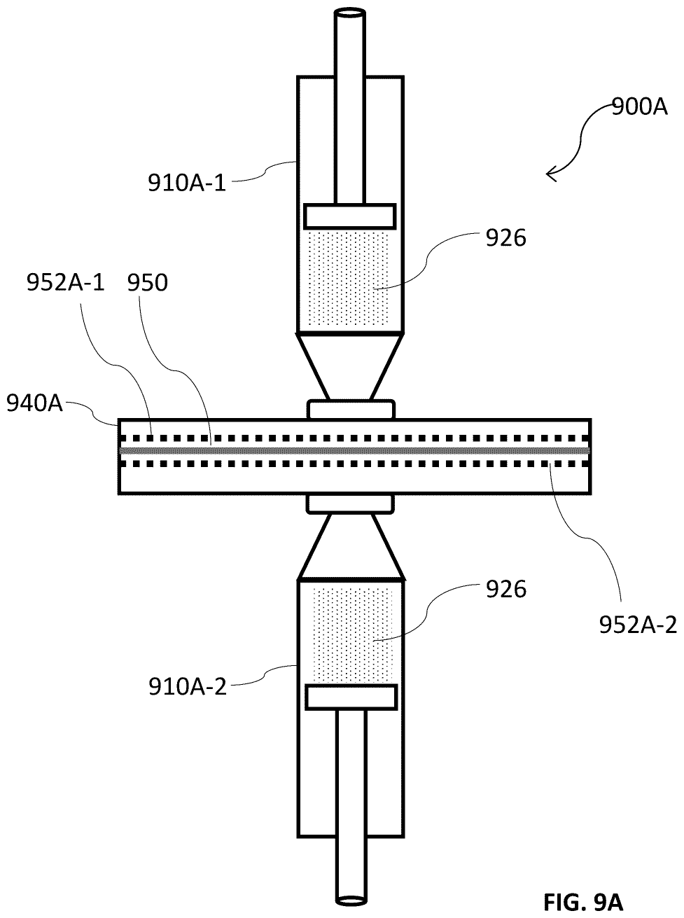

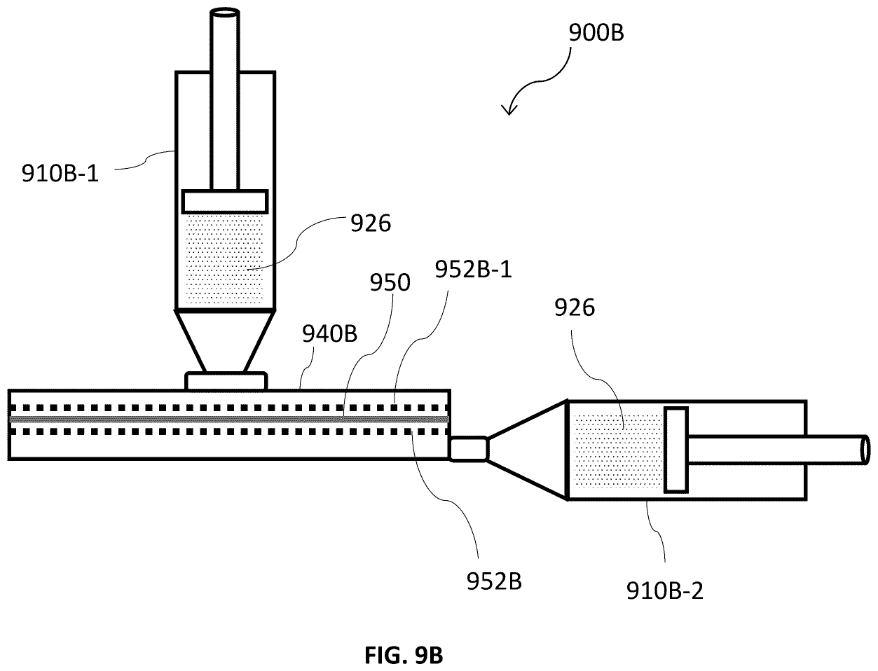

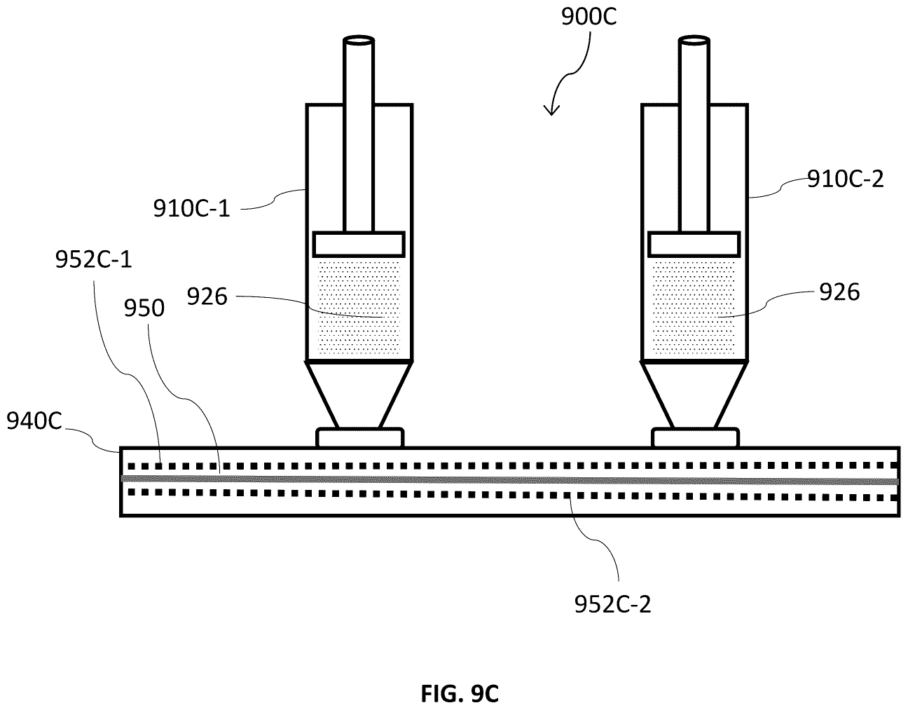

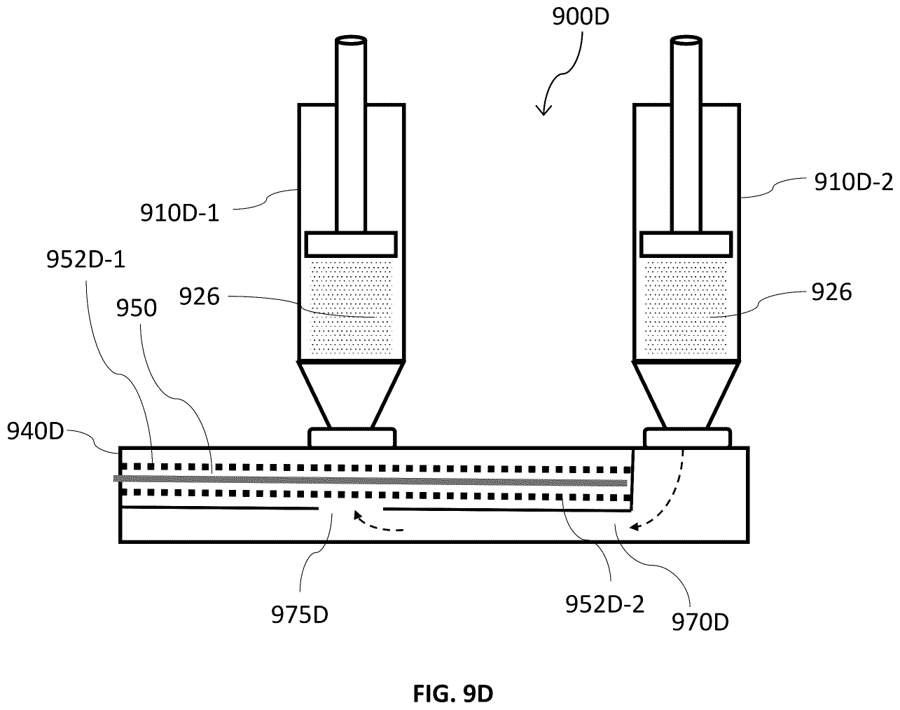

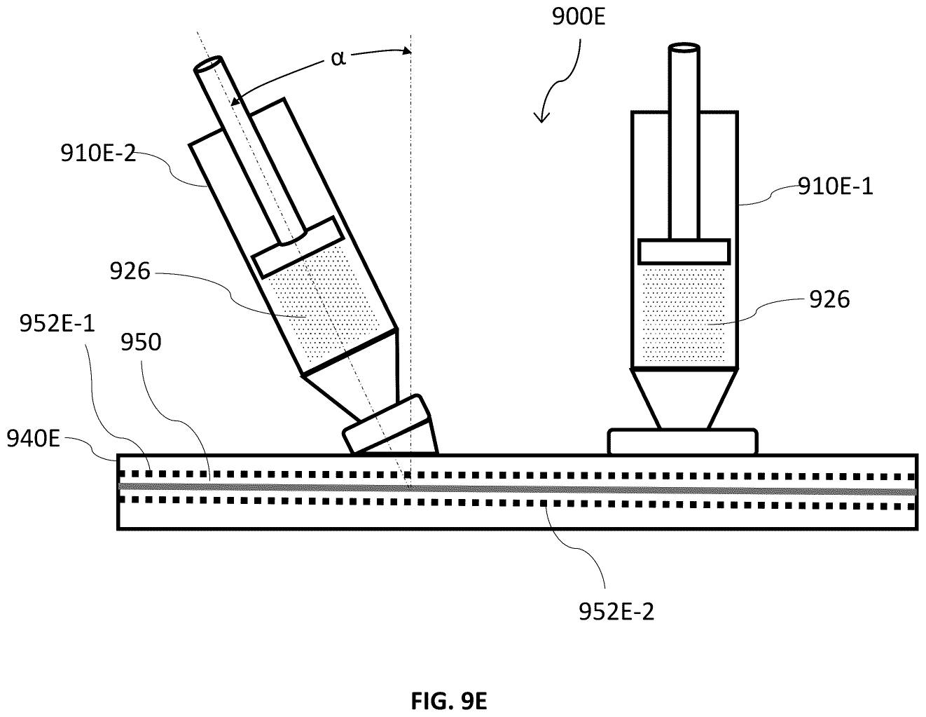

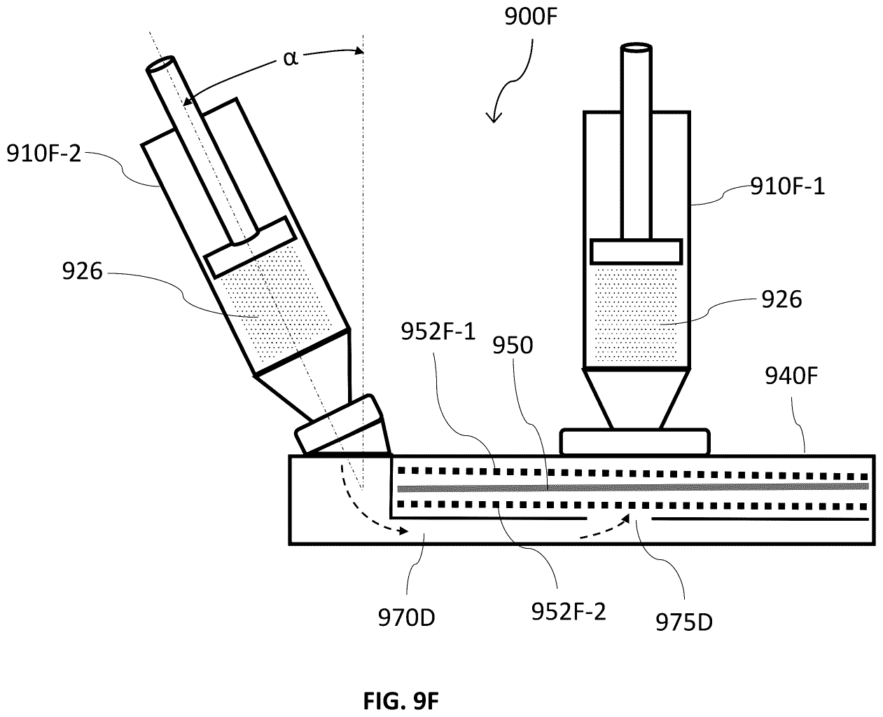

As shown in FIG. 9A, a system 900A may be configured to include two syringes 910A-1, 910A-2 or vacuum pumps arranged on opposite sides of a preparation chamber 940A to move a fat material across a skin graft material 950 contained in the preparation chamber 940A in a repeated manner, such as by drawing the fat material back and forth. A screen mesh 952A-1 may be provided in the preparation chamber 940A between the syringe 910A-1 and the skin graft material 950 and a screen mesh 952A-2 may be provided between the syringe 910B and the skin graft material 950. The screen mesh structures 952A-1, 952A-2 may be configured to retain the skin graft material 950 in the preparation chamber 940A against a positive or negative pressure therein, while allowing the fat material 926 to pass through. In this manner, the fat material 926 may impregnate the skin graft material 950, preparing a wound treatment according to the present disclosure. In some embodiments, the screen mesh 952A-1, 952A-2 may further separate individual components or portions of the fat material 926 as the fat material 926 is passed through the preparation chamber 940A, improving an application of the fat material 926 to or into the skin graft material 950.

In another related embodiment, as shown in FIG. 9B, a system 900B may be configured to include two syringes 910B-1, 910B-2 or vacuum pumps arranged perpendicularly to each other (or nearly perpendicularly to each other) on perpendicular sides of a preparation chamber 940B to move a fat material across a skin graft material 950 contained in a preparation chamber 940B in a repeated manner, such as by drawing the fat material back and forth. A screen mesh 952B-1 may be provided in the preparation chamber 940B between the syringe 910B-1 and the skin graft material 950 and a screen mesh 952B-2 may be provided between the syringe 910B-2 and the skin graft material 950. The screen mesh structures 952B-1, 952B-2 may be configured to retain the skin graft material 950 in the preparation chamber 940B against a positive or negative pressure therein, while allowing the fat material 926 to pass through. In this manner, the fat material 926 may impregnate the skin graft material 950, preparing a wound treatment according to the present disclosure. In some embodiments, the screen mesh 952B-1, 952B-2 may further separate individual components or portions of the fat material 926 as the fat material 926 is passed through the preparation chamber 940B, improving an application of the fat material 926 to or into the skin graft material 950.

In another related embodiment, as shown in FIG. 9C, a system 900C may be configured to include two syringes 910C-1, 910C-2 or vacuum pumps arranged the same side of a preparation chamber 940C to move a fat material across a skin graft material 950 contained in a preparation chamber 940C in a repeated manner, such as by drawing the fat material back and forth. A screen mesh 952C-1 may be provided in the preparation chamber 940C between the syringes 910C-1, 910C-2 and the skin graft material 950 and a screen mesh 952C-2 may be provided on an opposite side of the skin graft material 950 from the first screen mesh 952C-1. The screen mesh structures 952C-1, 952C-2 may be configured to retain the skin graft material 950 in the preparation chamber 940C against a positive or negative pressure therein, while allowing the fat material 926 to pass through. In this manner, the fat material 926 may impregnate the skin graft material 950 by operating the syringes 910C-1, 910C-2 in a repeated manner, thus preparing a wound treatment according to the present disclosure. In some embodiments, the screen mesh 952C-1, 952C-2 may further separate individual components or portions of the fat material 926 as the fat material 926 is passed through the preparation chamber 940C, improving an application of the fat material 926 to or into the skin graft material 950.

In another related embodiment, as shown in FIG. 9D, a system 900D may be configured to include two syringes 910D-1, 910D-2 or vacuum pumps arranged the same side of a preparation chamber 940D to move a fat material across a skin graft material 950 contained in a preparation chamber 940D in a repeated manner, such as by drawing the fat material back and forth. A screen mesh 952D-1 may be provided in the preparation chamber 940D between the syringe 910D-1 and the skin graft material 950 and a screen mesh 952D-2 may be provided on an opposite side of the skin graft material 950 from the first screen mesh 952D-1. The screen mesh structures 952D-1, 952D-2 may be configured to retain the skin graft material 950 in the preparation chamber 940D against a positive or negative pressure therein, while allowing the fat material 926 to pass through. In this embodiment, a channel 970D may optionally be provided for to facilitate passage of the fat material 926 injected by the second syringe 910D-2 to an underside of the skin graft material 950, as shown with the arrows showing movement of the fat material 926 to the underside of the skin graft material, through opening 975D. In this manner, the fat material 926 may impregnate the skin graft material 950 from both sides, preparing a wound treatment according to the present disclosure. In some embodiments, the screen mesh 952D-1, 952D-2 may further separate individual components or portions of the fat material 926 as the fat material 926 is passed through the preparation chamber 940D, improving an application of the fat material 926 to or into the skin graft material 950.

In another related embodiment, as shown in FIG. 9E, a system 900E may be configured to include two syringes 910E-1, 910E-2 or vacuum pumps arranged the same side of a preparation chamber 940E to move a fat material across a skin graft material 950 contained in a preparation chamber 940E in a repeated manner, such as by drawing the fat material back and forth. While syringe 910E-1 is arranged perpendicular or generally perpendicular to a top surface of the preparation chamber 940E, the second syringe 910E-2 is arranged offset from perpendicular to the top surface of the preparation chamber 940E by an angle a. A screen mesh 952E-1 may be provided in the preparation chamber 940E between the syringes 910E-1, 910E-2 and the skin graft material 950 and a screen mesh 952E-2 may be provided on an opposite side of the skin graft material 950 from the first screen mesh 952E-1. The screen mesh structures 952E-1, 952E-2 may be configured to retain the skin graft material 950 in the preparation chamber 940E against a positive or negative pressure therein, while allowing the fat material 926 to pass through. In this manner, the fat material 926 may impregnate the skin graft material 950 by operating the syringes 910E-1, 910E-2 in a repeated manner, thus preparing a wound treatment according to the present disclosure. In some embodiments, the screen mesh 952E-1, 952E-2 may further separate individual components or portions of the fat material 926 as the fat material 926 is passed through the preparation chamber 940E, improving an application of the fat material 926 to or into the skin graft material 950.

In another related embodiment, as shown in FIG. 9F, a system 900F may be configured to include two syringes 910F-1, 910F-2 or vacuum pumps arranged the same side of a preparation chamber 940F to move a fat material across a skin graft material 950 contained in a preparation chamber 940F in a repeated manner, such as by drawing the fat material back and forth. While syringe 910F-1 is arranged perpendicular or generally perpendicular to a top surface of the preparation chamber 940F, the second syringe 910F-2 is arranged offset from perpendicular to the top surface of the preparation chamber 940F by an angle a. A screen mesh 952F-1 may be provided in the preparation chamber 940F between the syringes 910F-1, 910F-2 and the skin graft material 950 and a screen mesh 952F-2 may be provided on an opposite side of the skin graft material 950 from the first screen mesh 952F-1. In this embodiment, a channel 970F may optionally be provided for to facilitate passage of the fat material 926 injected by the second syringe 910F-2 to an underside of the skin graft material 950, as shown with the arrows showing movement of the fat material 926 to the underside of the skin graft material, through opening 975F. The screen mesh structures 952F-1, 952F-2 may be configured to retain the skin graft material 950 in the preparation chamber 940F against a positive or negative pressure therein, while allowing the fat material 926 to pass through. In this manner, the fat material 926 may impregnate the skin graft material 950 by operating the syringes 910F-1, 910F-2 in a repeated manner, thus preparing a wound treatment according to the present disclosure. In some embodiments, the screen mesh 952F-1, 952F-2 may further separate individual components or portions of the fat material 926 as the fat material 926 is passed through the preparation chamber 940F, improving an application of the fat material 926 to or into the skin graft material 950.

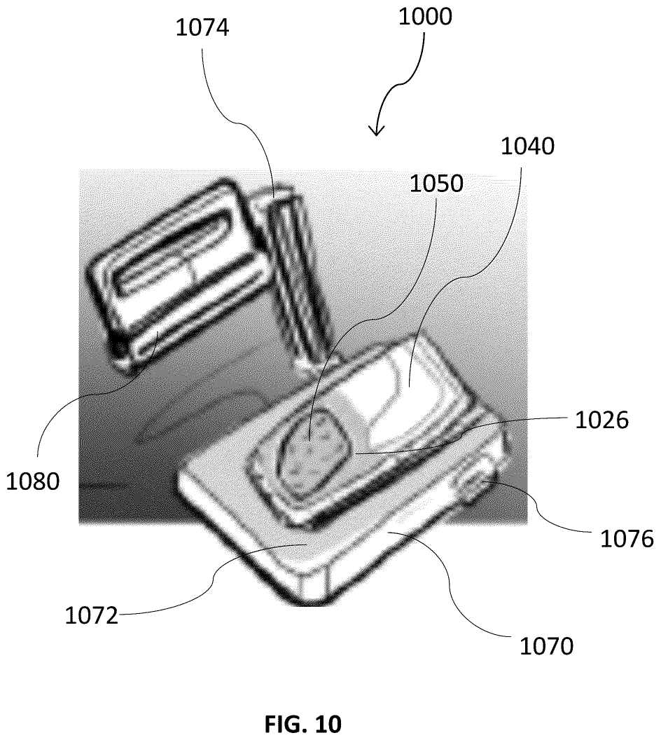

In another embodiment according to FIG. 10, a system 1000 for preparing a wound treatment may include a preparation chamber 1040, a supporting frame 1070 and a roller 1080. In this embodiment, a skin graft material 1050 and a fat material 1026 may be provided to the preparation chamber 1040 and the preparation chamber 1040 may be placed against the frame 1070. The roller 1080 may be placed against the preparation chamber 1040 opposite the frame 1070, such that the roller 1080 may roll along the preparation chamber 1040 and apply a pressure to the skin graft material 1050 and the fat material 1026 therein, in order to apply the fat material to the scaffold material 1050.

In some aspects, the roller 1080 may have a smooth surface or a patterned surface. For example, the roller 1080 may include a plurality of protrusions on the surface thereof in order to apply an irregular pressure across the preparation chamber 1040, such that the fat material 1026 may be broken into separate components and/or portions. The roller 1080 may include a handle or the like, such as for allowing a user to control the roller 1080 and apply a manual pressure thereto. In embodiments, an interior of the preparation chamber 1040 may include a patterned surface facing the skin graft material 1050. For example, the preparation chamber 1040 may have a plurality of protrusions on the interior surface thereof in order to apply an irregular pressure across the skin graft material 1050, such that the fat material 1026 may be broken into separate components and/or portions during application to the skin graft material 1050.

The frame 1070 in a simplest form may include a support surface 1072. In embodiments, the frame 1070 may include a retaining element 1074 in the form of a clip or clamp. In the depicted embodiment of FIG. 10, the retaining element may be movable between a first open configuration and a second closed configuration. In a closed configuration, the retaining element 1074 may be fixed by a cooperating locking element, such as a protrusion 1076, or another fixing means configured to interact with a corresponding portion of the retaining element 1074.

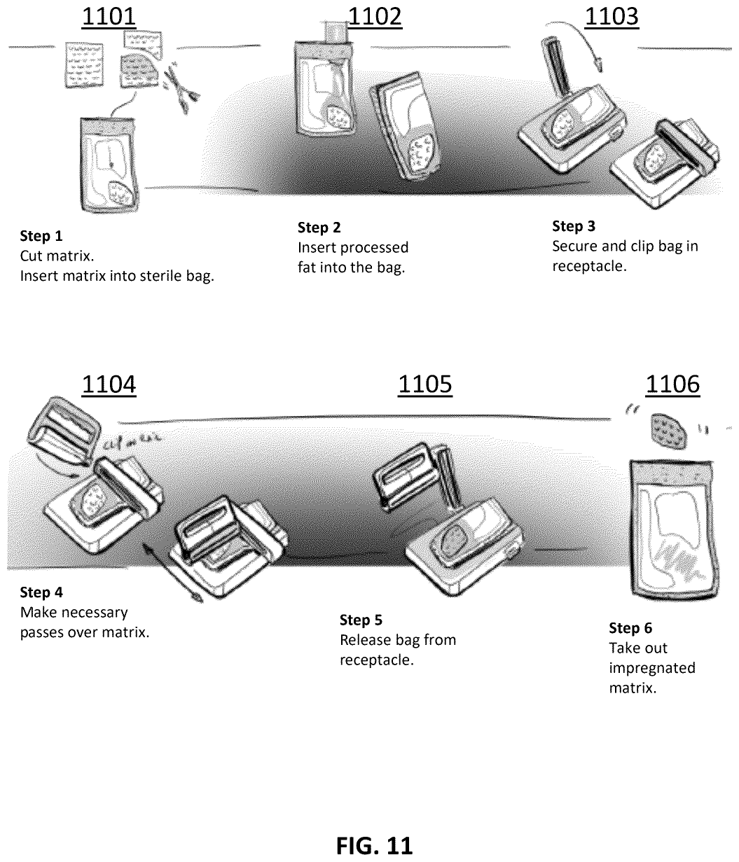

An embodiment of a method 1100 of using the system 1000 is depicted in FIG. 11. As shown, in a first step 1101 a skin graft material may be provided to a preparation chamber. In a second step 1102, a fat material may be added to the preparation chamber and then 1103 secured to a supporting frame, for example by fastening a clip connection between a retaining element and the supporting frame. A roller may then be passed over the preparation chamber in order to apply 1104 the fat material to the skin graft material. Subsequently, the preparation chamber may be released from the frame in step 1105 and the prepared skin graft material may be withdrawn from the preparation chamber at step 1106.

In varying aspects, the retaining element 1074 may comprise a roller, such that securing the preparation chamber 1040 to the frame 1070 allows the retaining element 1074 to perform both a retaining and rolling action when moved along the length of the frame 1070. In this manner, the system 1000 may form a mill or roller mill for compressing the skin graft material 1050 and the fat material 1026 in the preparation chamber 1040.

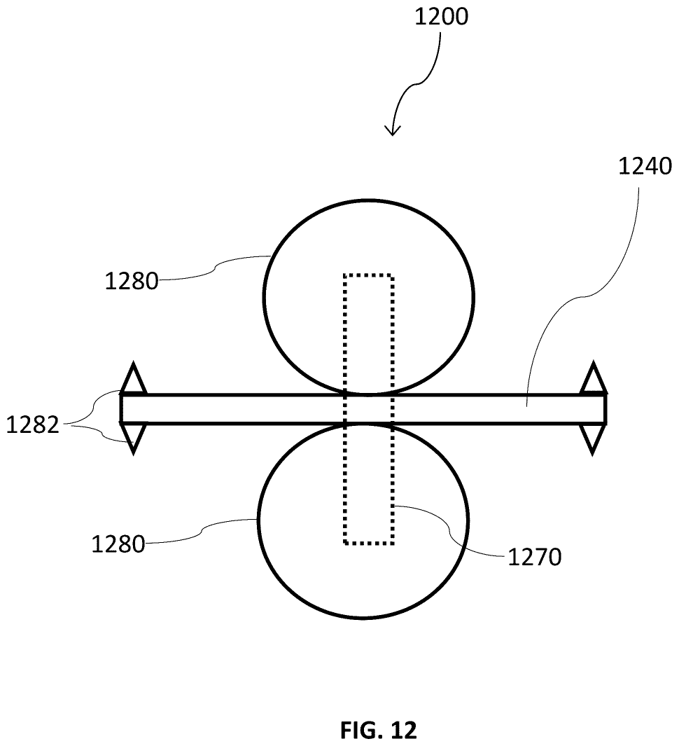

As illustrated in the diagram of FIG. 12, a system 1200 for preparing a wound treatment may include rollers 1280 and a preparation chamber 1240 enclosing a skin graft material and a fat material. The rollers 1280 may be configured to oppose each other across a predetermined distance, for example by mounting on a common or attachable frame 1270. The rollers 1280 may then be clipped or attached with the preparation chamber 1240 therebetween, such that the rollers 1280 may be passed back and forth across the preparation chamber 1240 similar to a rolling mill in metalworking.

In certain embodiments, the rollers 1280 and the preparation chamber 1240 may be provided with corresponding protrusions 1282 and/or grooves to limit movement of the preparation chamber 1240 to a predefined course between the rollers 1280. The protrusions 1282 and/or grooves may be provided in or on a frame of the preparation chamber 1240, such as in the form of a thickened portion having greater rigidity than the remaining portion of the preparation chamber 1240. In this manner, the preparation chamber may be more completely secured between the rollers 1280 and movement of the rollers 1280 over the preparation chamber 1240 may be performed in an easier way while retaining precise control over the movement of the rollers 1280 and/or the preparation chamber 1240.

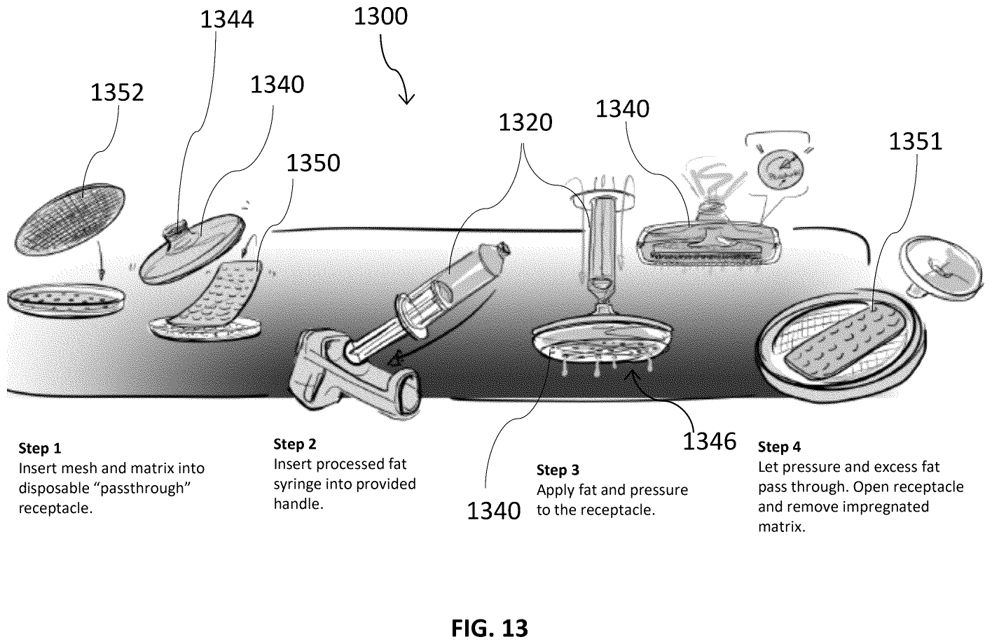

Referring to FIG. 13, a system 1300 for preparing a wound treatment may be provided with additional configurations. In the system 1300, a loading chamber 1320 containing a fat material may be connected to a preparation chamber 1340 retaining a skin graft material 1350 therein. The preparation chamber 1340 may include an input channel 1344 configured to cooperate with the loading chamber 1320. In certain examples, the loading chamber 1320 may comprise a syringe or syringe-like device, as illustrated. In embodiments, the preparation chamber 1340 may comprise cooperating elements that connect to enclose the skin graft material 1350 and a screen mesh 1352.

In the depicted embodiment of FIG. 13, a plurality of output channels 1346 may be provided in the preparation chamber 1340 opposite the input channel 1344. The screen mesh 1352 may be configured to retain the skin graft material 1350 in the preparation chamber 1340 against a positive pressure from the loading chamber 1320, while allowing the fat material to pass through. In embodiments, the fat material may impregnate the skin graft material 1350 and excess fat material may be forced out of the preparation chamber 1340 via the plurality of output channels 1346 and be collected or disposed of. The preparation chamber 1340 may then be opened and the prepared scaffold material 1351 removed for use as a wound treatment.

In some embodiments, the preparation chamber 1340 may comprise a rigid material and/or be reusable following cleaning and sterilization. A handle 1390 may be provided for receiving the loading chamber 1320, in order to allow a user more ergonometric support or leverage while using the loading chamber 1320. In certain embodiments, the handle 1390 may be configured to provide a predetermined resistance in order to reduce a pressure that may be applied to the fat material, or may be configured with assisting means, e.g., springs and the like, for increasing the pressure that may be applied to the fat material.

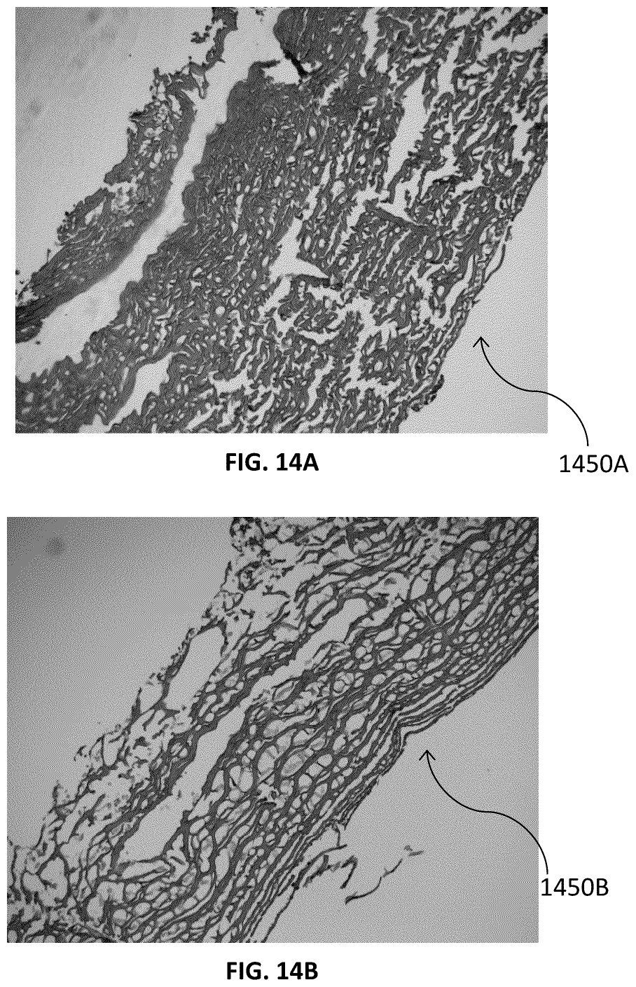

In some embodiments, additional advantages can be realized through a method of preparing a skin graft material for application of a fat material. In particular, it is possible that certain components of a fat material are too large to infiltrate, impregnate, infuse or join to a structure of existing scaffold materials, for example a collagen structure of decellularized fish skin. As shown in the hematoxylin and eosin staining images of decellularized fish skin scaffold materials that have been adequately lyophilized according to FIG. 14A and FIG. 14B, known methods for preparing skin graft materials 1450A, 1450B may result in tightly packed collagen structures having openings that are too small for adequate infiltration with a fat material according to the current disclosure. This may be due to the common belief that a more complete preservation of an ECM structure, including a pore size, in a skin graft material may lead to improved wound healing, while expansion or destruction of the pores is generally considered detrimental to wound healing.

For example, known scaffold materials may be frozen using liquid nitrogen or other special freezing equipment that can rapidly freeze the skin to −70° C. or lower, in order to preserve the collagen structure of the scaffold. In other common uses, scaffold materials may be lyophilized, i.e. frozen at a low temperature and under vacuum conditions so that water is removed sequentially from each ice crystal phase without ice re-crystallization, or cryopreserved, i.e. immersing the fish skin in a cryoprotectant solution prior to freezing, in order to preserve the collagen structure of the scaffold.

In contrast to known methods for preparing scaffold materials, methods of preparing a scaffold material for use a skin graft material according to the instant application may intentionally disrupt a collagen structure of the scaffold in order to facilitate impregnation of the scaffold material with a fat material, such as via annealing or fenestration of the scaffold material.

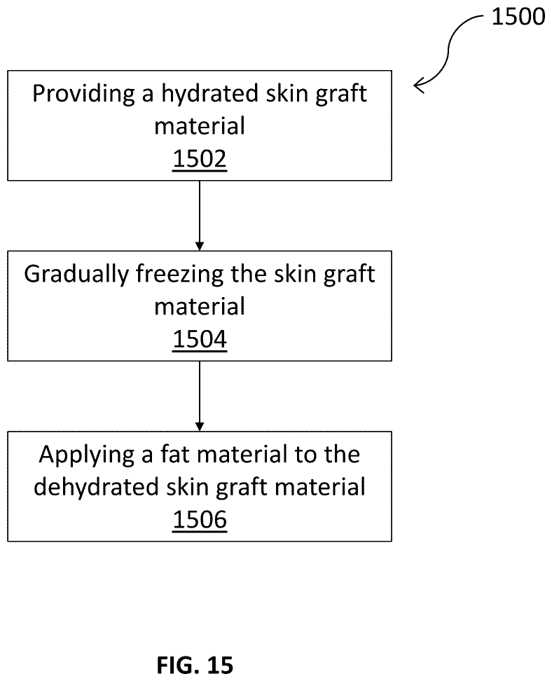

In a method 1500 according to an embodiment of FIG. 15, a scaffold material or skin graft material may be annealed such that a collagen structure of the scaffold material or skin graft material may be disrupted during lyophilization. In an embodiment, a hydrated scaffold material may be provided 1502, whether during an initial processing of a fish skin or a subsequent reprocessing. The hydrated scaffold material may then be frozen gradually 1504. For example, the hydrated scaffold material may be cooled to 0° C. and held at 0° C. for between 1 and 24hours, or between 2 and 12 hours, or between 3 and six hours. In this manner, pores in the collagen structure may be enlarged and the pore size may be balanced with a level of disruption of the ECM, such that the scaffold material retains a sufficiently intact ECM but with pore sizes large enough to facilitate impregnation with a dermal fat material. The freezing step may subsequently reach a temperature of −30° C. or lower, −40° C. or lower, or −70° C. or lower. During the freezing step, a pressure surrounding the scaffold material may be retained at atmospheric pressure or at a vacuum pressure being less than standard conditions for part or all of the freezing step. The resulting dehydrated scaffold material with an annealed collagen structure may then receive a fat material 1506, such as with a system according to embodiments of the current disclosure.

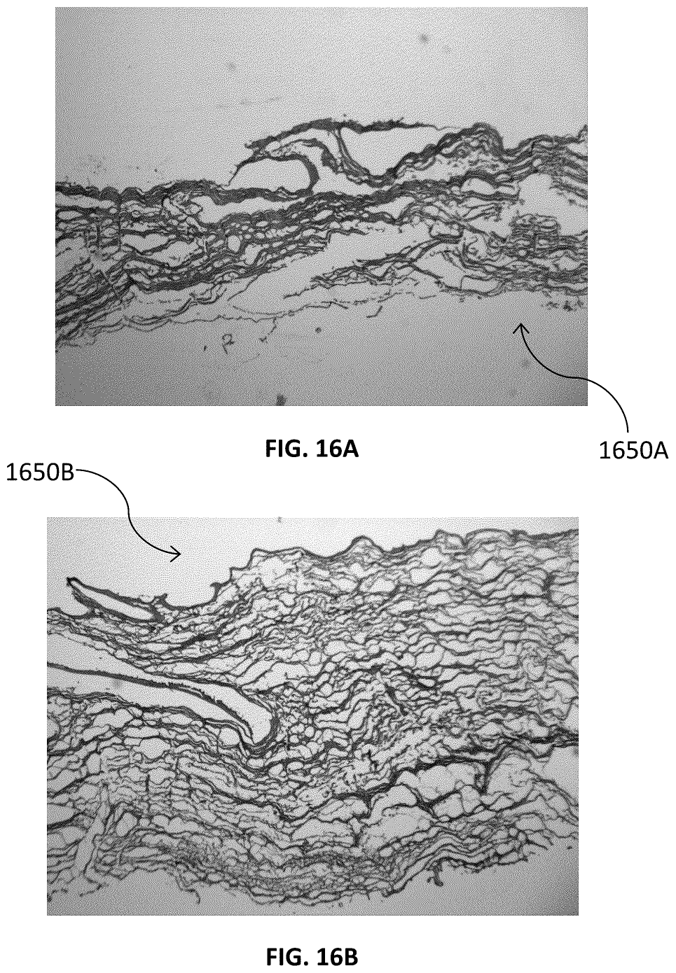

Surprisingly, as shown in the hematoxylin and eosin staining images of decellularized fish skin scaffold materials subjected to annealing according to FIG. 16A and FIG. 16B, a pore size of the scaffold materials 1650A, 1650B may be increased relative to the scaffold materials 1450A, 1450B while an ECM structure is largely retained. The increased pore size may then provide a new and unexpected and surprising advantage of allowing for improved impregnation or combination with a fat material according to the current disclosure. While previously believed to reduce the effectiveness of a skin graft material, an increased pore size of a skin graft material according to the described embodiments for use with a fat material has surprisingly been found by the inventors of the present application to improve wound healing, contrary to current expectations in the art.

A pore size of the annealed scaffold material may be configured to accommodate a fat material, for example including one or more of adipocytes, endothelial cells, fibroblasts, B and T-lymphocytes, macrophages, myeloid cells, pericytes, pre-adipocytes, smooth muscle cells, collagens, fibronectin, laminin, stromal cells, blood, lipids, debris from adipocyte rupture, and/or any surrounding biological components. According to varying embodiments of the disclosure, a skin graft material may be annealed such that the skin graft material includes pores having a size, dimension or diameter, for example an average diameter, in a range of 10 μm to 1000 μm, 20 μm to 600 μm, 40 μm to 300 μm, 80 μm to 300 μm, 100 μm to 500 μm, 20 μm to 100 μm, or 30 μm to 80 μm. In some embodiments, annealing a scaffold material may increase a pore size of the scaffold material by at least 50%, at least 100%, at least 150%, at least 200%, or at least 1000%.

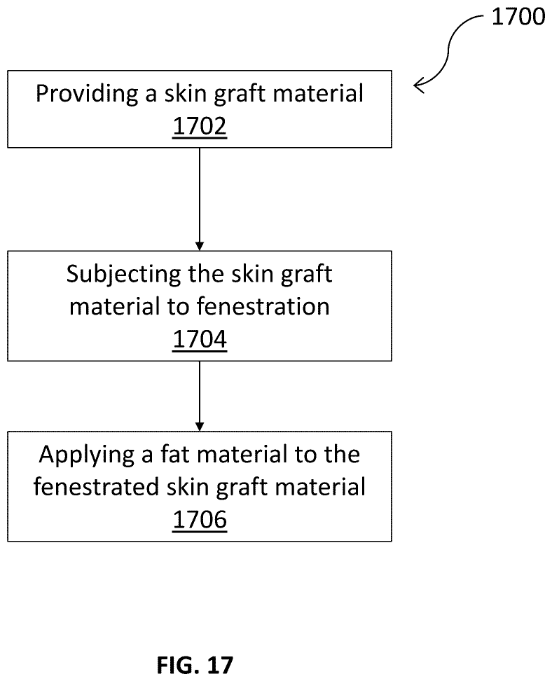

In another method 1700 according to an embodiment of FIG. 17, a scaffold material may be subjected to fenestration in order to disrupt a collagen structure of a scaffold material. In an embodiment, a scaffold material may be provided 1702, whether during an initial processing of a fish skin or a subsequent reprocessing. The scaffold material may then be fenestrated 1704 or cut to form a plurality of new openings in the scaffold material. The cuts may be made using in a micro fenestration procedure, such as using a computer-controlled cutting machine having a blade size of less than 2 mm or less than 1 mm. The fenestrated scaffold material may then receive a fat material 1706, such as with a system according to embodiments of the current disclosure. The fenestrated scaffold material may advantageously have a pore size greater than a starting scaffold material, as well as an increased surface area for application of the fat material. As such, an improved application of the fat material in the scaffold material may be achieved according to embodiments of the current disclosure.

Fenestration according to the current disclosure may include abrasion of a surface of the scaffold material or the skin graft material, in order to disrupt a surface of the scaffold material or skin graft material for application of a fat material. In some embodiments, fenestration may include the use of laser light energy or similar means for cutting or producing openings in the scaffold material or skin graft material.

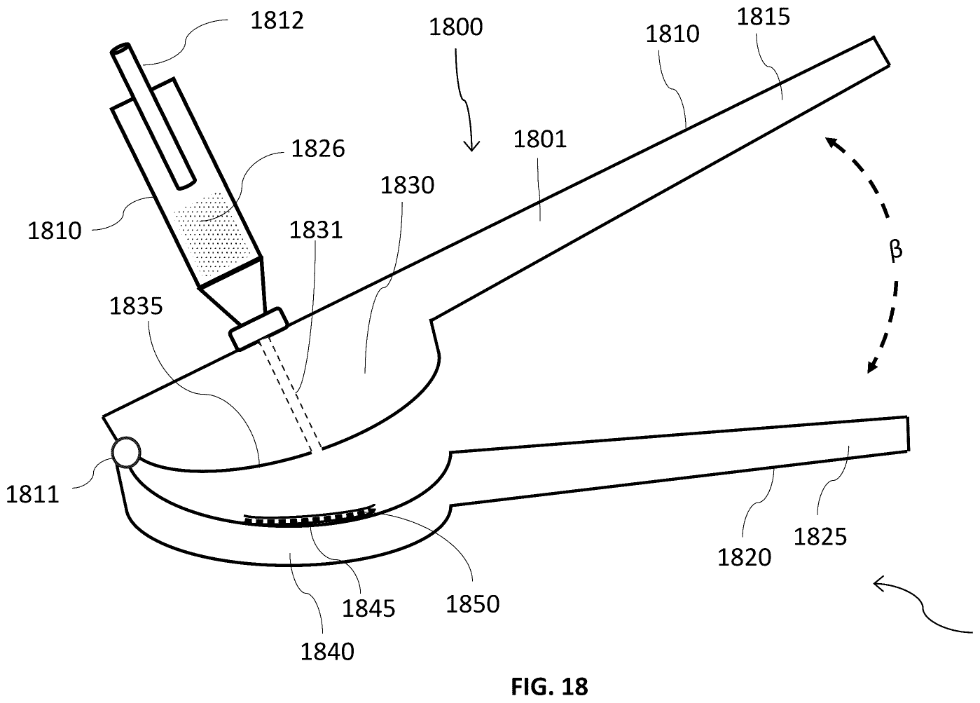

In another embodiment, as shown in FIG. 18, a system 1800 is shown for preparing a wound treatment in the form of a skin graft material with a fat material. The system includes a press 1801, which may be a hand-held press, having opposing sections, including a first section 1810 and an opposite section 1820. Handle portions 1815 and 1825, may be respectively provided on the first section 1810 and the second section 1820, the handle portions being configured to be held in the hand (or hands) or a medical practitioner, and may include a grip surface but more importantly provide leverage in the respective pivoting movement of the first and second sections 1810, 1820. Or alternatively, the handle may be actuated by a robotic mechanism. The opposing sections 1810, 1820 are allowed to pivot at a hinged fulcrum 1811 providing movement along the direction of arc B. Upper section 1810 is provided with a convex press portion having a convex press surface 1830 while lower section 1820 is provided with a concave press portion 1840 provided with a concave press surface 1845. Although in a different embodiment, the upper section could have the concave portion with a concave press surface and the lower section could have the convex press portion with a concave press surface. Or indeed, in another embodiment, the opposing press surfaces could indeed be parallel and planar, or generally parallel and planar.

In the embodiment of FIG. 18, a syringe 1810 is provided with fat material 1826 provided therein. Upper section 1810 further includes a narrow passage 1831 passing through the convex press portion 1830. A skin graft material 1850 may be placed within the press area, in this case, on the concave press surface 1845-although the opposing surface could also be configured to receive the skin graft material 1850. Upon or before closing the press 1801, the syringe plunger 1812 is actuated to cause the fat material 1826 to pass through passage 1831 and to contact the skin graft material 1850. And the opposing sections 1810, 1820 may be forced together applying a force to the fat material 1826 preparing a wound treatment according to the present disclosure. The opposing sections 1810, 1820 may be closed and opened in a related matter to force the fat material 1826 to penetrate the skin graft material 1850.

In some embodiments, additional advantages can be realized through the utilization of kits including systems of the current disclosure with corresponding skin graft materials. The kits may be provided as sterile, single-use kits for wound treatment. In use, a kit may be opened, the skin graft material cut or arranged to a desired shape, a fat material obtained from the patient, the fat material applied to the skin graft material using a system according to the disclosure, and the resulting skin graft material with fat material then applied to a wound.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

This disclosure provides various examples, embodiments, and features which improve a visual performance of a display and/or a camera recording an image from the display. Unless expressly stated, or unless such examples, embodiments, and features would be mutually exclusive, the various examples, embodiments, and features disclosed herein should be understood to be combinable with other examples, embodiments, or features described herein.

In addition to the above, further embodiments and examples include the following enumerated embodiments of displays and methods, including a first group of enumerated embodiments of displays with acoustic and/or light trap, and a second group of enumerated embodiments of displays and methods with stacked pixels, a third group of enumerated embodiments of displays and methods with macroblock with sync-banding (in 3D), and a fourth group of enumerated embodiments of displays and methods with multiplexing and staggered multiplexing. It is noted that the listed embodiments and examples of each of the four groups below can be combined with other listed embodiments and examples of the embodiments and examples of any, all, or a combination of the any or all the other groups.
1. A system for preparing a wound treatment, the system comprising: an applicator configured to apply a fat material to a skin graft material.
2. The system according to any or a combination of 1 above or 3-36 below, further comprising a preparation chamber configured to retain the skin graft material therein.
3. The system according to any or a combination of 1-2 above or 4-36 below, wherein the applicator comprises one or more of a syringe, a plunger, a roller, a screw, and a container.
4. The system according to any or a combination of 1-3 above or 5-36 below, wherein the applicator comprises an extrusion press.
5. The system according to any or a combination of 1-4 above or 6-36 below, wherein the extrusion press comprises:
   a loading chamber configured to receive a fat material via an input channel;
   a plunger at a first end of the loading chamber; and
   an extrusion die at a second end of the loading chamber opposite the first end;
   wherein the extrusion die connects the loading chamber and the preparation chamber, the plunger configured to force the fat material from the loading chamber, through the extrusion die, and into the preparation chamber.
6. The system according to any or a combination of 1-5 above or 7-36 below, wherein the input channel has an inner diameter of 4 mm or less.
7. The system according to any or a combination of 1-6 above or 8-36 below, wherein the input channel comprises an elastic material configured to substantially seal the input channel about a needle or cannula.
8. The system according to any or a combination of 1-7 above or 9-36 below, wherein the plunger has a diameter substantially equal to an interior diameter of the loading chamber.
9. The system according to any or a combination of 1-8 above or 10-36 below, wherein the extrusion die comprises one or more openings connecting the loading chamber to the preparation chamber, each of the one or more openings having a diameter of 1 mm or less.
10. The system according to any or a combination of 1-9 above or 11-36 below, wherein the extrusion die comprises one or more blades facing the plunger.
11. The system according to any or a combination of 1-10 above or 12-36 below, wherein the one or more blades define a profile of the one or more openings of the extrusion die.
12. The system according to any or a combination of 1-11 above or 13-36 below, wherein the one or more blades are provided in the one or more openings of the extrusion die.
13. The system according to any or a combination of 1-12 above or 14-36 below, wherein the preparation chamber comprises a retention element for securing the skin graft material.
14. The system according to any or a combination of 1-13 above or 15-36 below, wherein the retention element is configured to secure the skin graft material against the extrusion die.
15. The system according to any or a combination of 1-14 above or 16-36 below, further comprising:
   an output channel at a second side of the preparation chamber opposite the first side, the output channel configured to cooperate with a vacuum source; and
   a mesh screen provided in the preparation chamber;
   wherein the vacuum source is configured to generate a vacuum in the preparation chamber, such that the fat material is drawn from the loading chamber and through the skin graft material in the preparation chamber.
16. The system according to any or a combination of 1-15 above or 17-36 below, wherein the vacuum source comprises a peristaltic pump.
17. The system according to any or a combination of 1-16 above or 18-36 below, wherein the mesh screen has a screen size of 1 mm or less.
18. The system according to any or a combination of 1-17 above or 19-36 below, wherein the loading chamber comprises a syringe.
19. The system according to any or a combination of 1-18 above or 20-36 below, wherein the mesh screen is provided between the skin graft material and the output channel.
20. The system according to any or a combination of 1-19 above or 21-36 below, including a further mesh screen provided between the skin graft material and the input channel.
21. The system according to any or a combination of 1-20 above or 22-36 below, wherein the preparation chamber comprises a sealable bag.
22. The system according to any or a combination of 1-21 above or 23-36 below, wherein the output channel comprises a tube configured for use with a peristaltic pump.
23. The system according to any or a combination of 1-22 above or 24-36 below, wherein the mesh screen is larger than the skin graft material.
24. The system according to any or a combination of 1-23 above or 25-36 below, wherein the mesh screen has a varying screen size.
25. The system according to any or a combination of 1-24 above or 26-36 below, further comprising:
   a supporting frame; and
   a roller configured to apply a pressure against the preparation chamber opposite the supporting frame.
26. The system according to any or a combination of 1-25 above or 27-36 below, wherein the roller includes a handle for operating the roller.
27. The system according to any or a combination of 1-26 above or 28-36 below, wherein the supporting frame includes a retaining element for securing the preparation chamber thereto.
28. The system according to any or a combination of 1-27 above or 29-36 below, wherein the retaining element comprises a clamping arm that may be moved between a first open position and a second closed position, the clamping arm extending from a surface of the supporting frame.
29. The system according to any or a combination of 1-28 above or 30-36 below, wherein the roller comprises a plurality of projections on a surface thereof.
30. The system according to any or a combination of 1-29 above or 31-36 below, wherein the supporting frame is configured to secure the preparation chamber between the roller and a further opposing roller.
31. The system according to any or a combination of 1-30 above or 32-36 below, wherein the roller and the opposing roller are elastically secured to the frame, such that a distance between the roller and the opposing roller may vary based on a resistance provided by the preparation chamber.
32. The system according to any or a combination of 1-31 above or 33-36 below, wherein the mesh screen divides the preparation chamber into a first portion and a second portion.
33. The system according to any or a combination of 1-32 above or 34-36 below, comprising an opposing vacuum source opposite the vacuum source, the opposing vacuum source configured to generate an opposing vacuum in the preparation chamber, such that the fat material is drawn back through the skin graft material in the preparation chamber.
34. The system according to any or a combination of 1-33 above or 35-36 below, wherein the frame comprises a plurality of projections on a surface thereof.
35. The system according to any or a combination of 1-34 above or 36 below, wherein the preparation chamber comprises a sealable container.
36. The system according to any or a combination of 1-35 above, wherein the applicator includes a heating element and/or a heat conductive material.
37. A wound treatment kit comprising:
   a skin graft material; and
   an applicator configured to apply a fat material to the skin graft material.
38. The kit according to any or a combination of 37 above or 39-40 below, wherein the applicator comprises one or more of a syringe, an extrusion press, a loading chamber, a preparation chamber, and/or a pressure source.
39. The kit according to any or a combination of 37-38 above or 40 below, wherein one or more of the syringe, the extrusion press, the loading chamber, the preparation chamber, and/or the pressure source include fluid connections for fluidly connecting together.
40. The kit according to any or a combination of 37-39 above, further comprising a packaging enclosing at least the skin graft material and the applicator in a sterilized condition.
41. A wound treatment composition comprising:
   a skin graft material; and
   a fat material.
42. The composition according to any or a combination of 41 above or 43-60 below, wherein the skin graft material comprises a biologic material.
43. The composition according to any or a combination of 41-42 above or 44-60 below, wherein the biologic material comprises a fish skin.
44. The composition according to any or a combination of 41-43 above or 45-60 below, wherein the biologic material comprises a porcine skin.
45. The composition according to any or a combination of 41-44 above or 46-60 below, wherein the skin graft material comprises an extra cellular matrix material.
46. The composition according to any or a combination of 41-45 above or 47-60 below, wherein the skin graft material comprises a decellularized skin.
47. The composition according to any or a combination of 41-46 above or 48-60 below, wherein the skin graft material comprises a synthetic scaffold material.
48. The composition according to any or a combination of 41-47 above or 49-60 below, wherein the skin graft material comprises a plurality of comminuted particles.
49. The composition according to any or a combination of 41-48 above or 50-60 below, wherein the skin graft material has a pore size greater than an adipocyte.
50. The composition according to any or a combination of 41-49 above or 51-60 below, wherein the fat material comprises subcutaneous fat and/or visceral fat.
51. The composition according to any or a combination of 41-50 above or 52-60 below, wherein the fat material comprises dermal fat.
52. The composition according to any or a combination of 41-51 above or 53-60 below, wherein the fat material comprises lipoaspirate.
53. The composition according to any or a combination of 41-52 above or 54-60 below, wherein the fat material comprises a plurality of comminuted particles.
54. The composition according to any or a combination of 41-53 above or 55-60 below, wherein the skin graft material comprises a plurality of pores.
55. The composition according to any or a combination of 41-54 above or 56-60 below, wherein the pores of the skin graft material include the fat material therein.
56. The composition according to any or a combination of 41-55 above or 57-60 below, wherein the skin graft material is impregnated or infused with the fat material.
57. The composition according to any or a combination of 41-56 above or 58-60 below, wherein the fat material comprises at least 50% of a total weight of the composition.
58. The composition according to any or a combination of 41-57 above or 59-60 below, wherein the fat material comprises at least 50% of a total weight of the composition.
59. The composition according to any or a combination of 41-58 above or 60 below, wherein the skin graft material has a pore size greater than an adipocyte.
60. The composition according to any or a combination of 41-59 above, wherein the fat material comprises subcutaneous fat and/or visceral fat.
61. A method for preparing a wound treatment for a patient, the method comprising:
   providing a skin graft material;
   applying a fat material to the skin graft material.
62. The method according to any or a combination of 61 above or 63-80 below, wherein the step of providing the skin graft material comprises providing the skin graft material to a preparation chamber.
63. The method according to any or a combination of 61-62 above or 64-80 below, further comprising a step of preparing the skin graft material to receive the fat material.
64. The method according to any or a combination of 61-63 above or 65-80 below, wherein the step of preparing the skin graft material comprises increasing a pore size and/or surface area of the skin graft material by annealing, fenestration, and/or comminution.
65. The method according to any or a combination of 61-64 above or 66-80 below, wherein said annealing the skin graft material comprises freezing the skin graft material to form ice crystals therein.
66. The method according to any or a combination of 61-65 above or 67-80 below, wherein said freezing the skin graft material is performed at a temperature of 0° C. for between 1 and 24 hours.
67. The method according to any or a combination of 61-66 above or 68-80 below, wherein said freezing the skin graft material increases a pore size of the skin graft material.
68. The method according to any or a combination of 61-67 above or 69-80 below, wherein said fenestration comprises cutting the skin graft material with a blade of 1 mm or less.
69. The method according to any or a combination of 61-68 above or 70-80 below, wherein said fenestration comprises applying laser energy to the skin graft material to create openings therein.
70. The method according to any or a combination of 61-69 above or 71-80 below, wherein said fenestration comprises applying an abrasive to the skin graft material to create openings therein.
71. The method according to any or a combination of 61-70 above or 72-80 below, further comprising a step of obtaining the fat material.
72. The method according to any or a combination of 61-71 above or 73-80 below, wherein the step of obtaining the fat material comprises performing a liposuction procedure.
73. The method according to any or a combination of 61-72 above or 74-80 below, wherein the step of obtaining the fat material includes separating the fat material from a lipoaspirate of the liposuction procedure.
74. The method according to any or a combination of 61-73 above or 75-80 below, wherein the step of obtaining the fat material includes cutting the fat material to a predetermined size.
75. The method according to any or a combination of 61-74 above or 76-80 below, wherein the step of obtaining the fat material includes comminuting the fat material into particles of a predetermined size.
76. The method according to any or a combination of 61-75 above or 77-80 below, wherein the step of applying the fat material to the skin graft material comprises applying a pressure to the fat material and/or the skin graft material such that the fat material and the skin graft material are pressed or drawn together under said pressure.
77. The method according to any or a combination of 61-76 above or 78-80 below, wherein the step of applying the pressure to the fat material and/or the skin graft material includes applying the pressure for a predetermined period of time.
78. The method according to any or a combination of 61-77 above or 79-80 below, wherein the step of applying the fat material to the skin graft material comprises impregnating and/or infusing the skin graft material with the fat material.
79. The method according to any or a combination of 61-78 above or 80 below, wherein the step of applying the fat material to the skin graft material comprises infiltrating pores of the skin graft material with the fat material.
80. The method according to any or a combination of 61-79 above, wherein the step of applying the fat material to the skin graft material comprises applying heat energy to the fat material and/or the skin graft material.
81. A method for treating a wound, the method comprising:
   providing a skin graft material;
   applying a fat material to the skin graft material to form a combined fat and skin graft material; and applying the combined fat and skin graft material to the wound.
82. The method according to any or a combination of 81 above or 83-90 below, wherein the step of applying the combined fat and skin graft material to the wound occurs within 12 hours of the step of applying the fat material to the skin graft material.
83. The method according to any or a combination of 81-82 above or 84-90 below, wherein the step of applying the combined fat and skin graft material to the wound occurs within 12 hours of removing the fat material from a patient.
84. The method according to any or a combination of 81-83 above or 85-90 below, wherein the fat material is obtained from a patient having the wound.
85. The method according to any or a combination of 81-84 above or 86-00 below, further comprising a step of cutting the skin graft material to a shape of the wound.
86. The method according to any or a combination of 81-85 above or 87-90 below, wherein the skin graft material and/or the fat material comprise a plurality of comminuted particles, the step of applying the combined fat and skin graft material to the wound further comprising shaping the combined fat and skin graft material to fit the wound.
87. The method according to any or a combination of 81-86 above or 88-90 below, wherein the step of applying the combined fat and skin graft material to the wound comprises sealing the wound.
88. The method according to any or a combination of 81-87 above or 89-90 below, wherein the wound is sealed by a bandage and/or a suture at least partially covering the combined fat and skin graft material.
89. The method according to any or a combination of 81-88 above or 90 below, wherein the step of applying the combined fat and skin graft material to the wound occurs within 2 hours of the step of applying the fat material to the skin graft material.
90. The method according to any or a combination of 81-89 above, wherein the step of applying the combined fat and skin graft material to the wound occurs within 2 hours of removing the fat material from a patient.
91. A wound treatment composition comprising:
   a skin graft material having a plurality of pores,
   wherein the pores have an average diameter of 20 μm to 1000 μm, and
   optionally
      wherein the skin graft material has been treated to increase the pore size and/or surface area of the skin graft material by annealing, fenestration, and/or comminution,
      or
      wherein the skin graft material has been annealed to increase the pore size and/or surface area of the skin graft material by freezing the skin graft material to form ice crystals therein.
92. The composition according to any or a combination of 91 above or 93-100 below, wherein the skin graft material comprises a biologic material.
93. The composition according to any or a combination of 91-92 above or 94-100 below, wherein the biologic material comprises a fish skin.
94. The composition according to any or a combination of 91-93 above or 95-100 below, wherein the biologic material comprises a porcine skin.
95. The composition according to any or a combination of 91-95 above or 96-100 below, wherein the skin graft material comprises an extra cellular matrix material.
96. The composition according to any or a combination of 91-95 above or 97-100 below, wherein the skin graft material comprises a decellularized skin.
97. The composition according to any or a combination of 91-96 above or 98-100 below, wherein the skin graft material comprises a synthetic scaffold material.
98. The composition according to any or a combination of 91-97 above or 99-100 below, wherein the pores have an average diameter of 20 μm to 600 μm.
99. The composition according to any or a combination of 91-98 above or 100 below, wherein the pores have an average diameter of 20 μm to 300 μm.
100. The composition according to any or a combination of 91-99 above, wherein the pores have an average diameter of 20 μm to 100 μm.
101. A method for preparing a wound treatment for a patient, the method comprising:
   providing a skin graft material; and
   treating the skin graft material to increase a pore size and/or surface area of the skin graft material.
102. The method according to any or a combination of 101 above or 103-110 below, wherein the step of treating the skin graft material comprises increasing the pore size and/or surface area of the skin graft material by annealing, fenestration, and/or comminution.
103. The method according to any or a combination of 101-102 above or 104-110 below, wherein said annealing the skin graft material comprises freezing the skin graft material to form ice crystals therein.
104. The method according to any or a combination of 101-103 above or 105-110 below, wherein said freezing the skin graft material is performed at a temperature of 0° C. for between 1 and 24 hours.
105. The method according to any or a combination of 101-104 above or 106-110 below, wherein said freezing the skin graft material increases a pore size of the skin graft material.
106. The method according to any or a combination of 101-105 above or 107-110 below, wherein said fenestration comprises cutting the skin graft material with a blade of 1 mm or less.
107. The method according to any or a combination of 101-106 above or 108-110 below, wherein said fenestration comprises applying laser energy to the skin graft material to create openings therein.
108. The method according to any or a combination of 101-107 above or 109-110 below, wherein said fenestration comprises applying an abrasive to the skin graft material to create openings therein.
109. The method according to any or a combination of 101-108 above or 110 below, wherein the skin graft material comprises a biologic material.
110. The method according to any or a combination of 101-109 above, wherein treating the skin graft material increases an average diameter of the pores to within a range of 20 μm to 1000 μm.

The skilled artisan will recognize the interchangeability of various disclosed features. Besides the variations described herein, other known equivalents for each feature can be mixed and matched by one of ordinary skill in this art to prepare a wound treatment and utilize a method for making the same under principles of the present disclosure. The skilled artisan will understand that the features described herein may be adapted to other types of wound treatments and healthcare applications generally.

Although the system or method for preparing a wound treatment comprising a skin graft material provided with a fat material has been disclosed in certain preferred embodiments and examples, it therefore will be understood by those skilled in the art that the present disclosure extends beyond the disclosed embodiments to other alternative embodiments and/or uses of the system or method for preparing a wound treatment comprising a skin graft material provided with a fat material and obvious modifications and equivalents. It is intended that the scope of the present system or method for preparing a wound treatment comprising a skin graft material provided with a fat material disclosed should not be limited by the disclosed embodiments described above, but should be determined only by a fair reading of the claims that follow.

1-20. (canceled)

21. A system for preparing a wound treatment product, the system comprising an applicator configured to apply a fat material to a skin graft material.

22. The system according to claim 21, further comprising a preparation chamber configured to retain the skin graft material therein.

23. The system according to claim 21, wherein the applicator comprises one or more of a syringe, a plunger, a roller, a screw, and a container.

24. The system according to claim 21, wherein the applicator comprises an extrusion press.

25. The system according to claim 21, wherein the extrusion press comprises:
a loading chamber configured to receive a fat material via an input channel;
a plunger at a first end of the loading chamber; and
an extrusion die at a second end of the loading chamber opposite the first end;
wherein the extrusion die connects the loading chamber and the preparation chamber, the plunger configured to force the fat material from the loading chamber, through the extrusion die, and into the preparation chamber.

26. The system according to claim 21, wherein the extrusion die comprises one or more openings connecting the loading chamber to the preparation chamber, each of the one or more openings having a diameter of 1 mm or less.

27. The system according to claim 21, wherein the extrusion die comprises one or more blades facing the plunger, wherein the one or more blades define a profile of the one or more openings of the extrusion die or are provided in the one or more openings of the extrusion die.

28. The system according to claim 21, wherein the preparation chamber comprises a retention element for securing the skin graft material,
wherein the retention element is configured to secure the skin graft material against the extrusion die.

29. The system according to claim 21, further comprising:
an output channel at a second side of the preparation chamber opposite the first side, the output channel configured to cooperate with a vacuum source; and
a mesh screen provided in the preparation chamber;
wherein the vacuum source is configured to generate a vacuum in the preparation chamber, such that the fat material is drawn from the loading chamber and through the skin graft material in the preparation chamber.

30. The system according to claim 21, wherein the loading chamber comprises a syringe.

31. The system according to claim 21, wherein the preparation chamber comprises a sealable bag.

32. The system according to claim 21, further comprising:
a supporting frame; and
a roller configured to apply a pressure against the preparation chamber opposite the supporting frame.

33. The system according to claim 32, wherein the roller includes a handle for operating the roller.

34. The system according to claim 21, further comprising an opposing vacuum source opposite the vacuum source, the opposing vacuum source configured to generate an opposing vacuum in the preparation chamber, such that the fat material is drawn back through the skin graft material in the preparation chamber.

35. The system according to claim 22, wherein the applicator comprises a plurality of syringes, each configured to inject the fat material into the preparation chamber, and optionally each of the syringes being configured to withdraw the fat material from the preparation chamber.

36. The system according to claim 22, wherein the applicator includes two opposing press sections, including a first press section and a second press section configured to pivot relative to each other at a hinge point, each of the opposing press sections having a respective opposing press surface that define a preparation chamber, the opposing press surfaces providing a pressure to press the fat material into the skin graft material.

37. The system according to claim 36, wherein the opposing press surfaces include a concave press surface and a convex press surface.

38. The system according to claim 37, wherein the system includes a syringe provided on a back side of the first press section,
wherein the first press section includes a convex press section, and a passage is provided through the convex press section to provide passage of the fat material ejected from the syringe into the preparation chamber.

39. A method for preparing a skin graft material, the method comprising:
providing a skin graft material;
providing a system according to claim 21;
applying a fat material to the skin graft material using said system.

40. A wound treatment kit comprising:
a skin graft material; and
an applicator according to claim 21 configured to apply a fat material to the skin graft material.
